SEQUENCE LISTING (16 sequences)

SEQ ID NO: 1 - DNA, 3204 bp, Artificial Sequence
  HIV
atgggtgcccgagcttcggtactgtctggtggagagctggacagatgggagaaaattaggctgcgcccgggaggcaaaaagaaatacaagctcaagcatatcgtgtgggcctcgagggagcttgaacggtttgccgtgaacccaggcctgctggaaacatctgagggatgtcgccagatcctggggcaattgcagccatccctccagaccgggagtgaagagctgaggtccttgtataacacagtggctaccctctactgcgtacaccagaggatcgagattaaggataccaaggaggccttggacaaaattgaggaggagcaaaacaagagcaagaagaaggcccagcaggcagctgctgacactgggcatagcaaccaggtatcacagaactatcctattgtccaaaacattcagggccagatggttcatcaggccatcagcccccggacgctcaatgcctgggtgaaggttgtcgaagagaaggccttttctcctgaggttatccccatgttctccgctttgagtgagggggccactcctcaggacctcaatacaatgcttaataccgtgggcggccatcaggccgccatgcaaatgttgaaggagactatcaacgaggaggcagccgagtgggacagagtgcatcccgtccacgctggcccaatcgcgcccggacagatgcgggagcctcgcggctctgacattgccggcaccacctctacactgcaagagcaaatcggatggatgaccaacaatcctcccatcccagttggagaaatctataaacggtggatcatcctgggcctgaacaagatcgtgcgcatgtactctccgacatccatccttgacattagacagggacccaaagagccttttagggattacgtcgaccggttttataagaccctgcgagcagagcaggcctctcaggaggtcaaaaactggatgacggagacactcctggtacagaacgctaaccccgactgcaaaacaatcttgaaggcactaggcccggctgccaccctggaagagatgatgaccgcctgtcagggagtaggcggacccggacacaaagccagagtgttgatgggccccatcagtcccatcgagaccgtgccggtgaagctgaaacccgggatggacggccccaaggtcaagcagtggccactcaccgaggagaagatcaaggccctggtggagatctgcaccgagatggagaaagagggcaagatcagcaagatcgggccggagaacccatacaacacccccgtgtttgccatcaagaagaaggacagcaccaagtggcgcaagctggtggatttccgggagctgaataagcggacccaggatttctgggaggtccagctgggcatcccccatccggccggcctgaagaagaagaagagcgtgaccgtgctggacgtgggcgacgcttacttcagcgtccctctggacgaggactttagaaagtacaccgcctttaccatcccatctatcaacaacgagacccctggcatcagatatcagtacaacgtcctcccccagggctggaagggctctcccgccattttccagagctccatgaccaagatcctggagccgtttcggaagcagaaccccgatatcgtcatctaccagtacatggacgacctgtacgtgggctctgacctggaaatcgggcagcatcgcacgaagattgaggagctgaggcagcatctgctgagatggggcctgaccactccggacaagaagcatcagaaggagccgccattcctgaagatgggctacgagctccatcccgacaagtggaccgtgcagcctatcgtcctccccgagaaggacagctggaccgtgaacgacatccagaagctggtgggcaagctcaactgggctagccagatctatcccgggatcaaggtgcgccagctctgcaagctgctgcgcggcaccaaggccctgaccgaggtgattcccctcacggaggaagccgagctcgagctggctgagaaccgggagatcctgaaggagcccgtgcacggcgtgtactatgacccctccaaggacctgatcgccgaaatccagaagcagggccaggggcagtggacataccagatttaccaggagcctttcaagaacctcaagaccggcaagtacgcccgcatgaggggcgcccacaccaacgatgtcaagcagctgaccgaggccgtccagaagatcacgaccgagtccatcgtgatctgggggaagacacccaagttcaagctgcctatccagaaggagacctgggagacgtggtggaccgaatattggcaggccacctggattcccgagtgggagttcgtgaatacacctcctctggtgaagctgtggtaccagctcgagaaggagcccatcgtgggcgcggagacattctacgtggacggcgcggccaaccgcgaaacaaagctcgggaaggccgggtacgtcaccaaccggggccgccagaaggtcgtcaccctgaccgacaccaccaaccagaagacggagctgcaggccatctatctcgctctccaggactccggcctggaggtgaacatcgtgacggacagccagtacgcgctgggcattattcaggcccagccggaccagtccgagagcgaactggtgaaccagattatcgagcagctgatcaagaaagagaaggtctacctcgcctgggtcccggcccataagggcattggcggcaacgagcaggtcgacaagctggtgagtgcggggattagaaaggtgctgatggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctga

SEQ ID NO: 2 - protein, 1067 aa, Artificial Sequence
  HIV
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLMGPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLMVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNC

SEQ ID NO: 3 - DNA, 4665 bp, Artificial Sequence
  HIV
atggccgccagagccagcatcctgagcgggggcaagctggacgcctgggagaagatcagactgaggcctggcggcaagaagaagtaccggctgaagcacctggtgtgggccagcagagagctggatcgcttcgccctgaatcctagcctgctggagaccaccgagggctgccagcagatcatgaaccagctgcagcccgccgtgaaaaccggcaccgaggagatcaagagcctgttcaacaccgtggccaccctgtactgcgtgcaccagcggatcgacgtgaaggataccaaggaggccctggacaagatcgaggagatccagaacaagagcaagcagaaaacccagcaggccgctgccgacaccggcgacagcagcaaagtgagccagaactaccccatcatccagaatgcccagggccagatgatccaccagaacctgagccccagaaccctgaatgcctgggtgaaagtgatcgaggaaaaggccttcagccccgaagtgatccctatgttcagcgccctgagcgagggcgccaccccccaggacctgaacgtgatgctgaacattgtgggcggacaccaggccgccatgcagatgctgaaggacaccatcaatgaggaggccgccgagtgggacagactgcaccccgtgcaggccggacccatcccccctggccagatcagagagcccagaggcagcgacatcgccggcaccacctccacccctcaagaacagctgcagtggatgaccggcaaccctcccatccctgtgggcaacatctacaagcggtggatcatcctgggcctgaacaagattgtgcggatgtacagccccgtgtccatcctggatatcaagcagggccccaaggagcccttcagagactacgtggaccggttcttcaaggccctgagagccgagcaggccacccaggacgtgaagggctggatgaccgagaccctgctggtgcagaacgccaaccccgactgcaagagcatcctgaaggccctgggcagcggcgccacactggaggagatgatgaccgcctgccagggagtgggcggacccggccacaaggccagagtgctggccgaggccatgagccaggcccagcagaccaacatcatgatgcagcggggcaacttcagaggccagaagcggatcaagtgcttcaactgcggcaaggagggccacctggccagaaactgcagagcccccaggaagaagggctgctggaagtgtggcaaggaagggcaccagatgaaggactgcaccgagaggcaggccaatttcctgggcaagatttggcctagcagcaagggcagacccggcaatttcccccagagcagacccgagcccaccgcccctcccgccgagctgttcggcatgggcgagggcatcgccagcctgcccaagcaggagcagaaggacagagagcaggtgccccccctggtgtccctgaagtccctgttcggcaacgatcctctgagccagggatcccccatcagccccatcgagaccgtgcccgtgaccctgaagcccggcatggatggccccaaagtgaaacagtggcccctgaccgaggagaagattaaggccctgaccgaaatctgtaccgagatggagaaggagggcaagatcagcaagatcggccccgagaacccctacaacacccccatcttcgccatcaagaagaaggacagcaccaagtggcggaaactggtggacttccgggagctgaacaagaggacccaggacttctgggaagtgcagctgggcatcccccaccctgccggcctgaagaagaagaagtccgtgacagtgctggatgtgggcgacgcctacttcagcgtgcccctggacgagaacttcaggaagtacaccgccttcaccatccccagcaccaacaacgagacccccggagtgagataccagtacaacgtgctgcctcagggctggaagggcagccccgccatcttccagagcagcatgaccaagatcctggagcccttccggagcaagaaccccgagatcatcatctaccagtacatggccgccctgtatgtgggcagcgatctggagatcggccagcacaggaccaagatcgaagagctgagggcccacctgctgagctggggcttcaccacccccgataagaagcaccagaaggagccccctttcctgtggatgggctacgagctgcaccccgataagtggaccgtgcagcccatcatgctgcccgataaggagagctggaccgtgaacgacatccagaaactggtgggcaagctgaattgggccagccaaatctacgccggcattaaagtgaagcagctgtgcaggctgctgagaggcgccaaagccctgacagacatcgtgacactgacagaggaggccgagctggagctggccgagaacagggagatcctgaaggaccccgtgcacggcgtgtactacgaccccagcaaggacctggtggccgagattcagaagcagggccaggaccagtggacctaccaaatctaccaggagcctttcaagaacctgaaaaccgggaagtacgccaggaagagaagcgcccacaccaacgatgtgaggcagctggccgaagtggtgcagaaagtggctatggagagcatcgtgatctggggcaagacccccaagttcaagctgcccatccagaaggagacctgggaaacctggtggatggactactggcaggccacctggattcctgagtgggagttcgtgaacaccccccctctggtgaagctgtggtatcagctggagaaggaccccatcctgggcgccgagaccttctacgtggacggagccgccaatagagagaccaagctgggcaaggccggctacgtgaccgacagaggcagacagaaagtggtgtctctgaccgagacaaccaaccagaaaaccgagctgcacgccatcctgctggccctgcaggacagcggcagcgaagtgaacatcgtgaccgactcccagtacgccctgggcatcattcaggcccagcccgatagaagcgagagcgagctggtgaaccagatcatcgagaagctgatcggcaaggacaaaatctacctgagctgggtgcccgcccacaagggcatcggcggcaacgagcaggtggacaagctggtgtccagcggcatccggaaagtgctgtttctggacggcatcgacaaggcccaggaggaccacgagagataccacagcaactggcggacaatggccagcgacttcaacctgcctcccatcgtggccaaggagatcgtggccagctgcgataagtgtcagctgaagggcgaggccatgcacggccaggtggactgcagccctggcatctggcagctggcctgcacccacctggagggcaaagtgattctggtggccgtgcacgtggccagcggctacatcgaggccgaagtgattcccgccgagaccggccaggagaccgcctacttcctgctgaagctggccggcagatggcccgtgaaagtggtgcacaccgccaacggcagcaacttcacctctgccgccgtgaaggccgcctgttggtgggccaatatccagcaggagttcggcatcccctacaaccctcagagccagggcgtggtggccagcatgaacaaggagctgaagaagatcatcggccaggtgagggaccaggccgagcacctgaaaacagccgtgcagatggccgtgttcatccacaacttcaagcggaagggcggcattggcggctacagcgccggagagcggatcatcgacatcatcgccaccgatatccagaccaaggaactgcagaagcagatcaccaagattcagaacttcagagtgtactaccgggacagcagggaccccatctggaagggccctgccaagctgctgtggaagggcgaaggcgccgtggtgatccaggacaacagcgacatcaaagtggtgccccggaggaaggccaagattctgcgggactacggcaaacagatggccggcgatgactgcgtggccggcaggcaggatgaggacagatctatgggcggcaagtggtccaagggcagcattgtgggctggcccgagatccgggagagaatgagaagagcccctgccgccgctcctggagtgggcgccgtgtctcaggatctggataagcacggcgccatcaccagcagcaacatcaacaaccccagctgtgtgtggctggaggcccaggaagaggaggaagtgggcttccctgtgagaccccaggtgcccctgagacccatgacctacaagggcgccttcgacctgagccacttcctgaaggagaagggcggcctggacggcctgatctacagccggaagcggcaggagatcctggatctgtgggtgtaccacacccagggctacttccccgactggcagaattacacccctggccctggagtgcggtatcccctgaccttcggctggtgcttcaagctggtgcctatggagcccgacgaagtggagaaggccacagagggcgagaacaacagcctgctgcaccctatctgccagcacggcatggacgatgaggagcgggaagtgctgatctggaagttcgacagcaggctggccctgaagcacagagcccaggaactgcacccagagttctacaaggactgctga

SEQ ID NO: 4 - protein, 1554 aa, Artificial Sequence
  HIV
MAARASILSGGKLDAWEKIRLRPGGKKKYRLKHLVWASRELDRFALNPSLLETTEGCQQIMNQLQPAVKTGTEEIKSLFNTVATLYCVHQRIDVKDTKEALDKIEEIQNKSKQKTQQAAADTGDSSKVSQNYPIIQNAQGQMIHQNLSPRTLNAWVKVIEEKAFSPEVIPMFSALSEGATPQDLNVMLNIVGGHQAAMQMLKDTINEEAAEWDRLHPVQAGPIPPGQIREPRGSDIAGTTSTPQEQLQWMTGNPPIPVGNIYKRWIILGLNKIVRMYSPVSILDIKQGPKEPFRDYVDRFFKALRAEQATQDVKGWMTETLLVQNANPDCKSILKALGSGATLEEMMTACQGVGGPGHKARVLAEAMSQAQQTNIMMQRGNFRGQKRIKCFNCGKEGHLARNCRAPRKKGCWKCGKEGHQMKDCTERQANFLGKIWPSSKGRPGNFPQSRPEPTAPPAELFGMGEGIASLPKQEQKDREQVPPLVSLKSLFGNDPLSQGSPISPIETVPVTLKPGMDGPKVKQWPLTEEKIKALTEICTEMEKEGKISKIGPENPYNTPIFAIKKKDSTKWRKLVDFRELNKRTQDFWEVQLGIPHPAGLKKKKSVTVLDVGDAYFSVPLDENFRKYTAFTIPSTNNETPGVRYQYNVLPQGWKGSPAIFQSSMTKILEPFRSKNPEIIIYQYMAALYVGSDLEIGQHRTKIEELRAHLLSWGFTTPDKKHQKEPPFLWMGYELHPDKWTVQPIMLPDKESWTVNDIQKLVGKLNWASQIYAGIKVKQLCRLLRGAKALTDIVTLTEEAELELAENREILKDPVHGVYYDPSKDLVAEIQKQGQDQWTYQIYQEPFKNLKTGKYARKRSAHTNDVRQLAEVVQKVAMESIVIWGKTPKFKLPIQKETWETWWMDYWQATWIPEWEFVNTPPLVKLWYQLEKDPILGAETFYVDGAANRETKLGKAGYVTDRGRQKVVSLTETTNQKTELHAILLALQDSGSEVNIVTDSQYALGIIQAQPDRSESELVNQIIEKLIGKDKIYLSWVPAHKGIGGNEQVDKLVSSGIRKVLFLDGIDKAQEDHERYHSNWRTMASDFNLPPIVAKEIVASCDKCQLKGEAMHGQVDCSPGIWQLACTHLEGKVILVAVHVASGYIEAEVIPAETGQETAYFLLKLAGRWPVKVVHTANGSNFTSAAVKAACWWANIQQEFGIPYNPQSQGVVASMNKELKKIIGQVRDQAEHLKTAVQMAVFIHNFKRKGGIGGYSAGERIIDIIATDIQTKELQKQITKIQNFRVYYRDSRDPIWKGPAKLLWKGEGAVVIQDNSDIKVVPRRKAKILRDYGKQMAGDDCVAGRQDEDRSMGGKWSKGSIVGWPEIRERMRRAPAAAPGVGAVSQDLDKHGAITSSNINNPSCVWLEAQEEEEVGFPVRPQVPLRPMTYKGAFDLSHFLKEKGGLDGLIYSRKRQEILDLWVYHTQGYFPDWQNYTPGPGVRYPLTFGWCFKLVPMEPDEVEKATEGENNSLLHPICQHGMDDEEREVLIWKFDSRLALKHRAQELHPEFYKDC

SEQ ID NO: 5 - DNA, 2025 bp, Artificial Sequence
  HIV
atgagggtgatggagatccagcggaactgccagcacctgctgagatggggcatcatgatcctgggcatgattatcatctgcagcaccgccgacaacctgtgggtgaccgtgtactacggcgtgcctgtgtggagagatgccgagaccaccctgttctgcgccagcgacgccaaggcctacagcaccgagaagcacaatgtgtgggccacccacgcctgcgtgcctaccgatcccaaccctcaggagatccccctggacaacgtgaccgaggagttcaacatgtggaagaacaacatggtggaccagatgcacgaggacatcatcagcctgtgggaccagagcctgaagccctgcgtgcagctgacccccctgtgcgtgaccctgaactgcagcaacgccagagtgaacgccaccttcaactccaccgaggacagggagggcatgaagaactgcagcttcaacatgaccaccgagctgcgggataagaagcagcaggtgtacagcctgttctaccggctggacatcgagaagatcaacagcagcaacaacaacagcgagtaccggctggtgaactgcaataccagcgccatcacccaggcctgccctaaggtgaccttcgagcccatccccatccactactgcgcccctgccggcttcgccatcctgaagtgcaacgacaccgagttcaatggcaccggcccctgcaagaatgtgagcaccgtgcagtgcacccacggcatcaagcccgtggtgtccacccagctgctgctgaacggcagcctggccgagagagaagtgcggatcaggagcgagaacatcgccaacaacgccaagaacatcatcgtgcagttcgccagccccgtgaagatcaactgcatccggcccaacaacaatacccggaagagctacagaatcggccctggccagaccttctacgccaccgacattgtgggcgacatcagacaggcccactgcaacgtgtccaggaccgactggaacaacaccctgagactggtggccaaccagctgcggaagtacttcagcaacaagaccatcatcttcaccaacagcagcggcggagacctggagatcaccacccacagcttcaattgtggcggcgagttcttctactgcaacacctccggcctgttcaatagcacctggaccaccaacaacatgcaggagtccaacgacaccagcaacggcaccatcaccctgccctgccggatcaagcagatcatccggatgtggcagcgcgtgggccaggccatgtacgcccctcccatcgagggcgtgattcgctgcgagagcaacatcaccggcctgatcctgaccagagatggcggcaacaacaattccgccaacgagaccttcagacctggcggcggagatatccgggacaactggcggagcgagctgtacaagtacaaggtggtgaagatcgagcccctgggcgtggcccccaccagagccaagagaagagtggtggagcgggagaagagagccgtgggcatcggcgccgtgtttctgggcttcctgggagccgccggatctacaatgggagccgccagcatcaccctgaccgtgcaggccagacagctgctgagcggcatcgtgcagcagcagagcaatctgctgagagccatcgaggcccagcagcagctgctgaagctgacagtgtggggcatcaagcagctgcaggccagggtgctggccgtggagagatacctgagggaccagcagctcctgggcatctggggctgcagcggcaagctgatctgcaccaccaacgtgccctggaatagcagctggagcaacaagagctacgacgacatctggcagaacatgacctggctgcagtgggacaaggagatcagcaactacaccgacatcatctacagcctgatcgaggagagccagaaccagcaggagaagaacgagcaggatctgctggccctggacaagtgggccaacctgtggaactggttcgacatcagcaagtggctgtggtacatcagatcttga

SEQ ID NO: 6 - protein, 674 aa, Artificial Sequence
  HIV
MRVMEIQRNCQHLLRWGIMILGMIIICSTADNLWVTVYYGVPVWRDAETTLFCASDAKAYSTEKHNVWATHACVPTDPNPQEIPLDNVTEEFNMWKNNMVDQMHEDIISLWDQSLKPCVQLTPLCVTLNCSNARVNATFNSTEDREGMKNCSFNMTTELRDKKQQVYSLFYRLDIEKINSSNNNSEYRLVNCNTSAITQACPKVTFEPIPIHYCAPAGFAILKCNDTEFNGTGPCKNVSTVQCTHGIKPVVSTQLLLNGSLAEREVRIRSENIANNAKNIIVQFASPVKINCIRPNNNTRKSYRIGPGQTFYATDIVGDIRQAHCNVSRTDWNNTLRLVANQLRKYFSNKTIIFTNSSGGDLEITTHSFNCGGEFFYCNTSGLFNSTWTTNNMQESNDTSNGTITLPCRIKQIIRMWQRVGQAMYAPPIEGVIRCESNITGLILTRDGGNNNSANETFRPGGGDIRDNWRSELYKYKVVKIEPLGVAPTRAKRRVVEREKRAVGIGAVFLGFLGAAGSTMGAASITLTVQARQLLSGIVQQQSNLLRAIEAQQQLLKLTVWGIKQLQARVLAVERYLRDQQLLGIWGCSGKLICTTNVPWNSSWSNKSYDDIWQNMTWLQWDKEISNYTDIIYSLIEESQNQQEKNEQDLLALDKWANLWNWFDISKWLWYIRS

SEQ ID NO: 7 - DNA, 1545 bp, Artificial Sequence
  HIV
atgaaagtgaaggagaccaggaagaattatcagcacttgtggagatggggcaccatgctccttgggatgttgatgatctgtagtgctgcagaacaattgtgggtcacagtctattatggggtacctgtgtggaaagaagcaactaccactctattctgtgcatcagatgctaaagcatatgatacagaggtacataatgtttgggccacacatgcctgtgtacccacagaccccaacccacaagaagtagtattgggaaatgtgacagaatattttaacatgtggaaaaataacatggtagaccagatgcatgaggatataatcagtttatgggatcaaagcttgaagccatgtgtaaaattaaccccactctgtgttactttagattgcgatgatgtgaataccactaatagtactactaccactagtaatggttggacaggagaaataaggaaaggagaaataaaaaactgctcttttaatatcaccacaagcataagagataaggttcaaaaagaatatgcacttttttataaccttgatgtagtaccaatagatgatgataatgctactaccaaaaataaaactactagaaactttaggttgatacattgtaactcctcagtcatgacacaggcctgtccaaaggtatcatttgaaccaattcccatacattattgtgccccggctggttttgcgattctgaagtgtaacaataagacgtttgatggaaaaggactatgtacaaatgtcagcacagtacaatgtacacatggaattaggccagtagtgtcaactcaactgctgttaaatggcagtctagcagaagaagaggtagtaattagatctgacaatttcatggacaatactaaaaccataatagtacagctgaatgaatctgtagcaattaattgtacaagacccaacaacaatacaagaaaaggtatacatataggaccagggagagccttttatgcagcaagaaaaataataggagatataagacaagcacattgtaaccttagtagagcacaatggaataacactttaaaacagatagttataaaattaagagaacactttgggaataaaacaataaaatttaatcaatcctcaggaggggacccagaaattgtaaggcatagttttaattgtggaggggaatttttctactgtgatacaacacaactgtttaatagtacttggaatggtactgaaggaaataacactgaaggaaatagcacaatcacactcccatgtagaataaaacaaattataaacatgtggcaggaagtaggaaaagcaatgtatgcccctcccatcggaggacaaattagatgttcatcaaatattacagggctgctattaacaagagatggtggtaccgaagggaatgggacagagaatgagacagagatcttcagacctggaggaggagatatgagggacaattggagaagtgaattatataaatataaagtagtaaaagttgaaccactaggagtagcacccaccagggcaaagagaagagtggtgcagagataa

SEQ ID NO: 8 - protein, 514 aa, Artificial Sequence
  HIV
MKVKETRKNYQHLWRWGTMLLGMLMICSAAEQLWVTVYYGVPVWKEATTTLFCASDAKAYDTEVHNVWATHACVPTDPNPQEVVLGNVTEYFNMWKNNMVDQMHEDIISLWDQSLKPCVKLTPLCVTLDCDDVNTTNSTTTTSNGWTGEIRKGEIKNCSFNITTSIRDKVQKEYALFYNLDVVPIDDDNATTKNKTTRNFRLIHCNSSVMTQACPKVSFEPIPIHYCAPAGFAILKCNNKTFDGKGLCTNVSTVQCTHGIRPVVSTQLLLNGSLAEEEVVIRSDNFMDNTKTIIVQLNESVAINCTRPNNNTRKGIHIGPGRAFYAARKIIGDIRQAHCNLSRAQWNNTLKQIVIKLREHFGNKTIKFNQSSGGDPEIVRHSFNCGGEFFYCDTTQLFNSTWNGTEGNNTEGNSTITLPCRIKQIINMWQEVGKAMYAPPIGGQIRCSSNITGLLLTRDGGTEGNGTENETEIFRPGGGDMRDNWRSELYKYKVVKVEPLGVAPTRAKRRVVQR

SEQ ID NO: 9 - DNA, 2178 bp, Artificial Sequence
  Mycobacterium tuberculosis
atgcatcacacggccgcgtccgataacttccagctgtcccagggtgggcagggattcgccattccgatcgggcaggcgatggcgatcgcgggccagatccgatcgggtggggggtcacccaccgttcatatcgggcctaccgccttcctcggcttgggtgttgtcgacaacaacggcaacggcgcacgagtccaacgcgtggtcgggagcgctccggcggcaagtctcggcatctccaccggcgacgtgatcaccgcggtcgacggcgctccgatcaactcggccaccgcgatggcggacgcgcttaacgggcatcatcccggtgacgtcatctcggtgacctggcaaaccaagtcgggcggcacgcgtacagggaacgtgacattggccgagggacccccggccgaattcatggtggatttcggggcgttaccaccggagatcaactccgcgaggatgtacgccggcccgggttcggcctcgctggtggccgcggctcagatgtgggacagcgtggcgagtgacctgttttcggccgcgtcggcgtttcagtcggtggtctggggtctgacggtggggtcgtggataggttcgtcggcgggtctgatggtggcggcggcctcgccgtatgtggcgtggatgagcgtcaccgcggggcaggccgagctgaccgccgcccaggtccgggttgctgcggcggcctacgagacggcgtatgggctgacggtgcccccgccggtgatcgccgagaaccgtgctgaactgatgattctgatagcgaccaacctcttggggcaaaacaccccggcgatcgcggtcaacgaggccgaatacggcgagatgtgggcccaagacgccgccgcgatgtttggctacgccgcggcgacggcgacggcgacggcgacgttgctgccgttcgaggaggcgccggagatgaccagcgcgggtgggctcctcgagcaggccgccgcggtcgaggaggcctccgacaccgccgcggcgaaccagttgatgaacaatgtgccccaggcgctgcaacagctggcccagcccacgcagggcaccacgccttcttccaagctgggtggcctgtggaagacggtctcgccgcatcggtcgccgatcagcaacatggtgtcgatggccaacaaccacatgtcgatgaccaactcgggtgtgtcgatgaccaacaccttgagctcgatgttgaagggctttgctccggcggcggccgcccaggccgtgcaaaccgcggcgcaaaacggggtccgggcgatgagctcgctgggcagctcgctgggttcttcgggtctgggcggtggggtggccgccaacttgggtcgggcggcctcggtcggttcgttgtcggtgccgcaggcctgggccgcggccaaccaggcagtcaccccggcggcgcgggcgctgccgctgaccagcctgaccagcgccgcggaaagagggcccgggcagatgctgggcgggctgccggtggggcagatgggcgccagggccggtggtgggctcagtggtgtgctgcgtgttccgccgcgaccctatgtgatgccgcattctccggcagccggcgatatcgccccgccggccttgtcgcaggaccggttcgccgacttccccgcgctgcccctcgacccgtccgcgatggtcgcccaagtggggccacaggtggtcaacatcaacaccaaactgggctacaacaacgccgtgggcgccgggaccggcatcgtcatcgatcccaacggtgtcgtgctgaccaacaaccacgtgatcgcgggcgccaccgacatcaatgcgttcagcgtcggctccggccaaacctacggcgtcgatgtggtcgggtatgaccgcacccaggatgtcgcggtgctgcagctgcgcggtgccggtggcctgccgtcggcggcgatcggtggcggcgtcgcggttggtgagcccgtcgtcgcgatgggcaacagcggtgggcagggcggaacgccccgtgcggtgcctggcagggtggtcgcgctcggccaaaccgtgcaggcgtcggattcgctgaccggtgccgaagagacattgaacgggttgatccagttcgatgccgcgatccagcccggtgatgcgggcgggcccgtcgtcaacggcctaggacaggtggtcggtatgaacacggccgcgtcctag

SEQ ID NO: 10 - protein, 725 aa, Artificial Sequence
  Mycobacterium tuberculosis
MHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDAGGPVVNGLGQVVGMNTAAS

SEQ ID NO: 11 - DNA, 1149 bp, Artificial Sequence
  Plasmodium falciparum
atgatgagaaaacttgccatcctcagcgtcagctctttcctgttcgtggaggccctcttccaggagtatcagtgctacggaagcagcagcaatacaagggtcctgaacgagctcaactatgacaacgctggaacgaacctgtataacgagctggagatgaactactatggcaagcaggagaactggtatagcctgaagaagaacagccggtccctgggcgagaacgacgacggcaacaacaacaacggcgacaacggcagggagggcaaagatgaggacaagagggacgggaacaacgaggataacgagaagctgcggaagcccaagcacaagaaactcaagcagcccgccgacgggaacccggaccccaatgcaaatcccaacgtcgacccaaacgcaaaccctaacgtggaccccaacgccaatcccaacgtcgatcctaatgccaatccaaatgccaaccctaacgcaaatcctaatgcaaaccccaacgccaatcctaacgccaacccaaatgccaacccaaacgctaaccccaacgctaacccaaatgcaaatcccaatgctaacccaaacgtggaccctaacgctaaccccaacgcaaaccctaacgccaatcctaacgcaaaccccaatgcaaacccaaacgcaaatcccaacgctaaccctaacgcaaaccccaacgccaaccctaatgccaaccccaatgctaaccccaacgccaatccaaacgcaaatccaaacgccaacccaaatgcaaaccccaacgctaatcccaacgccaacccaaacgccaatcctaacaagaacaatcagggcaacgggcagggccataacatgccgaacgaccctaatcggaatgtggacgagaacgccaacgccaacagcgccgtgaagaacaacaacaacgaggagccctccgacaagcacatcaaggaatacctgaacaagatccagaacagtctgagcaccgagtggtccccctgctccgtgacctgcggcaacggcatccaggtgaggatcaagcccggctccgccaacaagcccaaggacgagctggactacgccaacgacatcgagaagaagatctgcaagatggagaaatgcagctctgtgttcaacgtcgtgaactccgccatcggcctgtga

SEQ ID NO: 12 - protein, 382 aa, Artificial Sequence
  Plasmodium falciparum
MMRKLAILSVSSFLFVEALFQEYQCYGSSSNTRVLNELNYDNAGTNLYNELEMNYYGKQENWYSLKKNSRSLGENDDGNNNNGDNGREGKDEDKRDGNNEDNEKLRKPKHKKLKQPADGNPDPNANPNVDPNANPNVDPNANPNVDPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSAIGL

SEQ ID NO: 13 - DNA, 1275 bp, Artificial Sequence
  Plasmodium falciparum
atgatggctcccgatcctaatgcaaatccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacagtgctgtaaaaaataataataacgaagaaccaagtgataagcacataaaagaatatttaaacaaaatacaaaattctctttcaactgaatggtccccatgtagtgtaacttgtggaaatggtattcaagttagaataaagcctggctctgctaataaacctaaagacgaattagattatgcaaatgatattgaaaaaaaaatttgtaaaatggaaaaatgttccagtgtgtttaatgtcgtaaatagttcaataggattagggcctgtgacgaacatggagaacatcacatcaggattcctaggacccctgctcgtgttacaggcggggtttttcttgttgacaagaatcctcacaataccgcagagtctagactcgtggtggacttctctcaattttctagggggatcacccgtgtgtcttggccaaaattcgcagtccccaacctccaatcactcaccaacctcctgtcctccaatttgtcctggttatcgctggatgtgtctgcggcgttttatcatattcctcttcatcctgctgctatgcctcatcttcttattggttcttctggattatcaaggtatgttgcccgtttgtcctctaattccaggatcaacaacaaccaatacgggaccatgcaaaacctgcacgactcctgctcaaggcaactctatgtttccctcatgttgctgtacaaaacctacggatggaaattgcacctgtattcccatcccatcgtcctgggctttcgcaaaatacctatgggagtgggcctcagtccgtttctcttggctcagtttactagtgccatttgttcagtggttcgtagggctttcccccactgtttggctttcagctatatggatgatgtggtattgggggccaagtctgtacagcatcgtgagtccctttataccgctgttaccaattttcttttgtctctgggtatacatttaa

SEQ ID NO: 14 - protein, 424 aa, Artificial Sequence
  Plasmodium falciparum
MMAPDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLGPVTNMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTNTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 15 - DNA, 3411 bp, Artificial Sequence
  HIV
atggtcattgttcagaacatacagggccaaatggtccaccaggcaattagtccgcgaactcttaatgcatgggtgaaggtcgtggaggaaaaggcattctccccggaggtcattccgatgttttctgcgctatctgagggcgcaacgccgcaagaccttaataccatgcttaacacggtaggcgggcaccaagccgctatgcaaatgctaaaagagactataaacgaagaggccgccgaatgggatcgagtgcacccggtgcacgccggcccaattgcaccaggccagatgcgcgagccgcgcgggtctgatattgcaggaactacgtctacccttcaggagcagattgggtggatgactaacaatccaccaatcccggtcggagagatctataagaggtggatcatactgggactaaacaagatagtccgcatgtattctccgacttctatactggatatacgccaaggcccaaaggagccgttcagggactatgtcgaccgattctataagacccttcgcgcagagcaggcatcccaggaggtcaaaaattggatgacagaaactcttttggtgcagaatgcgaatccggattgtaaaacaattttaaaggctctaggaccggccgcaacgctagaagagatgatgacggcttgtcagggagtcggtggaccggggcataaagcccgcgtcttacacatgggcccgatatctccgatagaaacagtttcggtcaagcttaaaccagggatggatggtccaaaggtcaagcagtggccgctaacggaagagaagattaaggcgctcgtagagatttgtactgaaatggagaaggaaggcaagataagcaagatcgggccagagaacccgtacaatacaccggtatttgcaataaagaaaaaggattcaacaaaatggcgaaagcttgtagattttagggaactaaacaagcgaacccaagacttttgggaagtccaactagggatcccacatccagccggtctaaagaagaagaaatcggtcacagtcctggatgtaggagacgcatattttagtgtaccgcttgatgaggacttccgaaagtatactgcgtttactataccgagcataaacaatgaaacgccaggcattcgctatcagtacaacgtgctcccgcagggctggaaggggtctccggcgatatttcagagctgtatgacaaaaatacttgaaccattccgaaagcagaatccggatattgtaatttaccaatacatggacgatctctatgtgggctcggatctagaaattgggcagcatcgcactaagattgaggaactgaggcaacatctgcttcgatggggcctcactactcccgacaagaagcaccagaaggagccgccgttcctaaagatgggctacgagcttcatccggacaagtggacagtacagccgatagtgctgcccgaaaaggattcttggaccgtaaatgatattcagaaactagtcggcaagcttaactgggcctctcagatttacccaggcattaaggtccgacagctttgcaagctactgaggggaactaaggctctaacagaggtcatcccattaacggaggaagcagagcttgagctggcagagaatcgcgaaattcttaaggagccggtgcacggggtatactacgacccctccaaggaccttatagccgagatccagaagcaggggcagggccaatggacgtaccagatatatcaagaaccgtttaagaatctgaagactgggaagtacgcgcgcatgcgaggggctcatactaatgatgtaaagcaacttacggaagcagtacaaaagattactactgagtctattgtgatatggggcaagaccccaaagttcaagctgcccatacagaaggaaacatgggaaacatggtggactgaatattggcaagctacctggattccagaatgggaatttgtcaacacgccgccacttgttaagctttggtaccagcttgaaaaggagccgatagtaggggcagagaccttctatgtcgatggcgccgcgaatcgcgaaacgaagctaggcaaggcgggatacgtgactaataggggccgccaaaaggtcgtaacccttacggataccaccaatcagaagactgaactacaagcgatttaccttgcacttcaggatagtggcctagaggtcaacatagtcacggactctcaatatgcgcttggcattattcaagcgcagccagatcaaagcgaaagcgagcttgtaaaccaaataatagaacagcttataaagaaagagaaggtatatctggcctgggtccccgctcacaagggaattggcggcaatgagcaagtggacaagctagtcagcgctgggattcgcaaggttcttgcgatggggggtaagtggtctaagtctagcgtagtcggctggccgacagtccgcgagcgcatgcgacgcgccgaaccagccgcagatggcgtgggggcagcgtctagggatctggagaagcacggggctataacttccagtaacacggcggcgacgaacgccgcatgcgcatggttagaagcccaagaagaggaagaagtagggtttccggtaactccccaggtgccgttaaggccgatgacctataaggcagcggtggatctttctcacttccttaaggagaaaggggggctggagggcttaattcacagccagaggcgacaggatattcttgatctgtggatttaccatacccaggggtactttccggactggcagaattacaccccggggccaggcgtgcgctatcccctgactttcgggtggtgctacaaactagtcccagtggaacccgacaaggtcgaagaggctaataagggcgagaacacttctcttcttcacccggtaagcctgcacgggatggatgacccagaacgagaggttctagaatggaggttcgactctcgacttgcgttccatcacgtagcacgcgagctgcatccagaatatttcaagaactgccgcccaatgggcgccagggccagtgtacttagtggcggagaactagatcgatgggaaaagatacgcctacgcccggggggcaagaagaagtacaagcttaagcacattgtgtgggcctctcgcgaacttgagcgattcgcagtgaatccaggcctgcttgagacgagtgaaggctgtaggcaaattctggggcagctacagccgagcctacagactggcagcgaggagcttcgtagtctttataataccgtcgcgactctctactgcgttcatcaacgaattgaaataaaggatactaaagaggcccttgataaaattgaggaggaacagaataagtcgaaaaagaaggcccagcaggccgccgccgacaccgggcacagcaaccaggtgtcccaaaactactaa

SEQ ID NO: 16 - protein, 1136 aa, Artificial Sequence
  HIV
MVIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLHMGPISPIETVSVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSCMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLAMGGKWSKSSVVGWPTVRERMRRAEPAADGVGAASRDLEKHGAITSSNTAATNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCRPMGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNY

(19) United States
(12) Patent Application Publication
(10) Pub. No.: US 2010/0055166 A1
(43) Pub. Date: Mar. 4, 2010

(54) NOVEL METHOD AND COMPOSITIONS

(76) Inventor: Gerald Hermann Voss, Rixensart (BE)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/529,062

(22) PCT Filed: Feb. 28, 2008
(86) PCT No.: PCT/EP2008/052448

§ 371 Date: Aug. 28, 2009

Related U.S. Application Data
(60) Provisional application No. 60/892,714, filed on Mar. 2, 2007.

(51) Int. Cl. A61K 9/127; A61K 39/235; A61K 39/295; A61P 37/04

(52) U.S. Cl. 424/450; 424/233.1; 424/201.1

(57) ABSTRACT

The present invention relates to, inter alia, a method of raising an immune response against a pathogen which comprises administering (i) one or more first immunogenic polypeptides derived from said pathogen; (ii) one or more adenoviral vectors comprising one or more heterologous polynucleotides encoding one or more second immunogenic polypeptides derived from said pathogen; and (iii) an adjuvant; wherein the one or more first immunogenic polypeptides, the one or more adenoviral vectors and the adjuvant are administered concomitantly. The invention also relates to vaccines, pharmaceutical compositions, kits and uses employing said polypeptides, adenoviral vectors and adjuvants.

### FIELD OF THE INVENTION

This invention relates to novel vaccine compositions and their use in the stimulation of immune responses in mammals, especially humans, and in particular for the prevention and treatment of infection by pathogens. In particular it relates to compositions capable of inducing CD4+ and CD8+ T-cell responses as well as antibody responses in subjects without recourse to complex prime-boost schedules.

### BACKGROUND TO THE INVENTION

Inactivated whole organisms have been used in successful vaccination since the late nineteenth century. In more recent times, vaccines involving the administration of extracts, subunits, toxoids and capsular polysaccharides have been employed. Since genetic engineering techniques have been available, the use of recombinant proteins has been a favoured strategy, obviating many of the risks associated with use of purified proteins from natural sources.

Early vaccine approaches were based on the administration of proteins which stimulated some aspect of the immune response in vivo. Subsequently it was appreciated that immune responses could also be raised by administration of DNA which could be transcribed and translated by the host into an immunogenic protein.

The mammalian immune response has two key components: the humoral response and the cell-mediated response. The humoral response involves the generation of circulating antibodies which will bind to the antigen to which they are specific, thereby neutralising the antigen and favouring its subsequent clearance by a process involving other cells that are either cytotoxic or phagocytic. B-cells are responsible for generating antibodies (plasma B cells), as well as holding immunological humoral memory (memory B-cells), i.e. the ability to recognise an antigen some years after first exposure to it eg through vaccination. The cell mediated response involves the interplay of numerous different types of cells, among which are the T cells. T-cells are divided into a number of different subsets, mainly the CD4+ and CD8+ T cells.

Antigen-presenting cells (APC) such as macrophages and dendritic cells act as sentinels of the immune system, screening the body for foreign antigens. When extracellular foreign antigens are detected by APC, these antigens are phagocytosed (engulfed) inside the APC where they will be processed into smaller peptides. These peptides are subsequently presented on major histocompatibility complex class II (MHC II) molecules at the surface of the APC where they can be recognised by antigen-specific T lymphocytes expressing the CD4 surface molecules (CD4+ T cells). When CD4+ T cells recognise the antigen to which they are specific on MHCII molecules in the presence of additional adequate co-stimulatory signals, they become activated and secrete an array of cytokines that subsequently activate the other arms of the immune system. In general, CD4+ T cells are classified into T helper 1 (Th1) or T helper 2 (Th2) subsets depending on the type of response they generate following antigen recognition. Upon recognition of a peptide-MHC II complex, Th1 CD4+ T cells secrete interleukins and cytokines such as interferon gamma thereby activating macrophages to release toxic chemicals such as nitric oxide and reactive oxygen/nitrogen species. IL-2 and TNF-alpha are also commonly categorized as Th1 cytokines. In contrast, Th2 CD4+ T cells generally secrete interleukins such as IL-4, IL-5 or IL-13.

Other functions of the T helper CD4+ T cells include providing help to activate B cells to produce and release antibodies. They can also participate to the activation of antigen-specific CD8+ T cells, the other major T cell subset beside CD4+ T cells.

CD8+ T cells recognize the peptide to which they are specific when it is presented on the surface of a host cell by major histocompatibility class I (MHC I) molecules in the presence of appropriate costimulatory signals. In order to be presented on MHC I molecules, a foreign antigen need to directly access the inside of the cell (the cytosol or nucleus) such as it is the case when a virus or intracellular bacteria directly penetrate a host cell or after DNA vaccination. Inside the cell, the antigen is processed into small peptides that will be loaded onto MHC I molecules that are redirected to the surface of the cell. Upon activation CD8+ T cells secrete an array of cytokines such as interferon gamma that activates macrophages and other cells. In particular, a subset of these CD8+ T cells secretes lytic and cytotoxic molecules (e.g. granzyme, perforin) upon activation. Such CD8+ T cells are referred to as cytotoxic T cells.

More recently, an alternative pathway of antigen presentation involving the loading of extracellular antigens or fragments thereof onto MHCI complexes has been described and called “cross-presentation”.

The nature of the T-cell response is also influenced by the composition of the adjuvant used in a vaccine. For instance, adjuvants containing MPL & QS21 have been shown to activate Th1 CD4+ T cells to secrete IFN-gamma (Stewart et al. Vaccine. 2006, 24 (42-43):6483-92).

Whereas adjuvants are well known to have value in enhancing immune responses to protein antigens, they have not generally been used in conjunction with DNA or DNA-based vector vaccination. There are several hypotheses as to why adjuvants have not been used in conjunction with DNA-vector based vaccines. Indeed, interferences between the adjuvant and the vector may have an impact on their stability. In addition, one might expect that adding an adjuvant to an attenuated vector could increase the reactogenicity induced by such product. Finally, increasing the immunogenicity of a DNA-vector based vaccine may lead to an enhanced neutralizing immune response against the vector itself, thereby precluding any boosting effect of subsequent injections of the same vector-based vaccine. In fact, in a vaccination protocol directed towards protection against P. falciparum infection, Jones et al (2001, J Infect Diseases 183, 303-312) have reported an adverse outcome after combining DNA, recombinant protein and adjuvant as a boosting composition following a prime by DNA. Indeed, the levels of parasitemia were significantly lower in a group in which the boosting composition contained protein and adjuvant only. It was concluded that use of the combination of DNA, recombinant protein and adjuvant in this protocol adversely affected the outcome on parasitemia as well as antibody responses.

On the other hand, there has been a report of enhancement of the efficacy of an adjuvanted DNA-based vector vaccine (Ganne et al. Vaccine (1994) 12(13) 1190-1196). In particular, the enhanced efficacy of a replication-defective adenovirus-vectored vaccine by the addition of oil adjuvants was correlated with higher antibody levels but the impact on CD4 and CD8 T cell responses was not reported.

The use of an apathogenic virus as an adjuvant has been disclosed in WO2007/016715. It was not mentioned that said virus could contain any heterologous polynucleotide.

It is generally thought that stimulation of both CD4+ and CD8+ cells are needed for optimal protective immunity, especially in certain diseases such as HIV infection/AIDS. In order to induce an optimal immune response either prophylactically or therapeutically, stimulation of both CD4+ and CD8+ cells is desirable. This is one of the main goal of “prime-boost” vaccination strategies in which the alternate administration of protein-based vaccines (inducing mostly CD4+ T cells) with DNA-vector based vaccines, i.e. naked DNA, viral vectors or intracellular bacterial vectors such as listeria, (inducing mostly CD8+ T cells) or vice versa most likely activates both CD4+ and CD8+ T cell responses.

However, although prime-boost vaccine strategies may generally give rise to a greater or more balanced response, the requirement to vaccinate on more than one occasion and certainly on more than two occasions can be burdensome or even unviable, especially in mass immunization programs for the developing world.

Furthermore, as already mentioned above, it is often not possible to boost the viral vector component because of immunity that may have been raised against the vector itself.

Thus the objects of the invention include one or more of the following: (a) to provide a complete vaccination protocol and a vaccine composition which stimulates the production of CD4+ and/or CD8+ cells and/or antibodies and in particular which obviates or mitigates the need for repeated immunizations; (b) to provide a vaccination protocol and a vaccine composition which better stimulates production of CD4+ cells and/or CD8+ cells and/or antibodies relative to vaccine compositions containing an immunogenic polypeptide alone or a polynucleotide alone or relative to a conventional prime-boost protocol involving separate administration of immunogenic polypeptide and polynucleotide; (c) to provide a vaccine composition which stimulates or better stimulates Th1 responses; (d) to provide a vaccine composition and vaccination protocol in which required doses of components, especially viral vectors, are minimised; and (e) more generally to provide a useful vaccine composition and vaccination protocol for treatment or prevention of diseases caused by pathogens. By “better stimulates” is meant that the intensity and/or persistence of the response is enhanced.

### SUMMARY OF THE INVENTION

Thus according to the invention there is provided a method of raising an immune response against a pathogen which comprises administering (i) one or more First immunogenic polypeptides derived from said pathogen; (ii) one or more adenoviral vectors comprising one or more heterologous polynucleotides encoding one or more second immunogenic polypeptides derived from said pathogen; and (iii) an adjuvant; wherein the one or more first immunogenic polypeptides, the one or more adenoviral vectors and the adjuvant are administered concomitantly.

According to a specific aspect of the invention there is provided a vaccine composition comprising (i) one or more first immunogenic polypeptides derived from a pathogen; (ii) one or more adenoviral vectors comprising one or more heterologous polynucleotide encoding one or more second immunogenic polypeptides derived from said pathogen; and (iii) an adjuvant.

There is also provided an immunogenic composition comprising (i) one or more first immunogenic polypeptides derived from a pathogen; (ii) one or more adenoviral vectors comprising one or more heterologous polynucleotides encoding one or more second immunogenic polypeptides derived from said pathogen; and (iii) an adjuvant.

Said vaccines and immunogenic compositions suitably stimulate production of pathogen-specific CD4+ T-cells and/or CD8+ T-cells and/or antibodies.

By “pathogen-specific CD4+ T-cells and/or CD8+ T-cells and/or antibodies” is meant CD4+ T-cells and/or CD8+ T-cells and/or antibodies which specifically recognise the whole pathogen or a part (eg an immunogenic subunit) thereof. By “specifically recognise” is meant that the CD4+ T-cells and/or CD8+ T-cells and/or antibodies recognise in an immunospecific rather than a non-specific manner said pathogen (or part thereof).

There is also provided a method of stimulating an immune response in a mammal which comprises administering to a subject an immunologically effective amount of such a composition.

There is also provided use of such a composition in the manufacture of a medicament for stimulating an immune response in a mammal.

There is also provided such a composition for use in stimulating an immune response in a mammal.

There is also provided a method of stimulating the production of pathogen-specific CD4+ T-cells and/or CD8+ T-cells and/or antibodies in mammals which comprises administering to said mammal (i) one or more first immunogenic polypeptides derived from a pathogen; (ii) one or more adenoviral vectors comprising one or more heterologous polynucleotides encoding one or more second immunogenic polypeptides derived from said pathogen; and (iii) an adjuvant; wherein the one or more first immunogenic polypeptides, the one or more adenoviral vectors and the adjuvant are administered concomitantly, for example by administering an immunologically effective amount of an aforeseaid composition.

There is also provided use of aforesaid compositions in the manufacture of a medicament for stimulating the production of pathogen specific CD4+ and/or CD8+ cells and/or antibodies in mammals.

For example, production of CD4+ T-cells or CD8+ T-cells or antibodies is stimulated.

Suitably production of 2 and especially 3 of CD4+ T-cells and/or CD8+ T-cells and/or antibodies is stimulated.

Suitably production of CD8+ T-cells is stimulated. Suitably production of CD4+ and CD8+ T-cells is stimulated. Suitably production of CD4+ and CD8+ T-cells and antibodies is stimulated.

Alternatively suitably production of CD4+ T-cells is stimulated. Suitably production of CD4+ and antibodies is stimulated.

Alternatively suitably production of antibodies is stimulated.

The methods of the invention are suitably intended to provide the steps adequate for a complete method for raising an immune response (although the method may, if desired, be repeated). Therefore suitably the methods do not involve use of a priming dose of any immunogenic polypeptide or polynucleotide (e.g. in the form of a vector such as an adenoviral vector) encoding any immunogenic polypeptide.

For example there is provided a method of raising an immune response against a pathogen which consists of (a) administering (i) one or more first immunogenic polypeptides derived from said pathogen; (ii) one or more adenoviral vectors comprising one or more heterologous polynucleotides encoding one or more second immunogenic polypeptides derived from said pathogen; and (iii) an adjuvant; wherein the one or more immunogenic polypeptide, the one or more adenoviral vector and the adjuvant are administered concomitantly; and (b) optionally repeating the steps of (a).

The steps of the method may be repeated (e.g. repeated once) if a repeat gives rise to an improved immune response. An adequate response, at least as far as a T-cell response is concerned, may be obtained without any need for repetition.

There is also provided a method of raising an immune response against a pathogen which comprises (a) administering (i) one or more first immunogenic polypeptides derived from said pathogen; (ii) one or more adenoviral vectors comprising one or more heterologous polynucleotides encoding one or more second immunogenic polypeptides derived from said pathogen; and (iii) an adjuvant; wherein the one or more first immunogenic polypeptides, the one or more adenoviral vectors and the adjuvant are administered concomitantly; and wherein the method does not involve administering any priming dose of immunogenic polypeptide or polynucleotide encoding immunogenic polypeptide.

There is also provided a kit comprising (i) one or more first immunogenic polypeptides derived from a pathogen; (ii) one or more adenoviral vectors comprising one or more heterologous polynucleotides encoding one or more second immunogenic polypeptides derived from said pathogen; and (iii) an adjuvant; and in particular comprising (i) one or more first immunogenic polypeptides derived from a pathogen and an adjuvant; and (ii) one or more second adenoviral vectors comprising one or more heterologous polynucleotides encoding one or more immunogenic polypeptides derived from said pathogen; for use in a method according to the invention.

Compositions and methods of the invention may be useful for the prevention of infection by pathogens in naïve subjects, or prevention of re-infection in subjects who have previously been infected by pathogen or treatment of subjects who have been infected by pathogen.

### BRIEF DESCRIPTION OF THE FIGURES

FIG. 1 shows a graphical representation of the construction of plasmid p73i-Tgrn

FIGS. 2-8 show the results of experiments discussed in Example 1, specifically:

FIGS. 2a, 2b, 3a, 3b: CD4+ and CD8+ T-cell responses in response to restimulation by pools of peptides derived from p24, RT, Nef and p17 following various immunization protocols and at different timepoints;

FIG. 4: antibody responses against F4;

FIGS. 5-8 antibody responses against F4 components p24, RT, p17 and Nef respectively;

FIG. 9 shows the results of experiments discussed in Example 2, specifically:

CD4+ T-cell responses in response to restimulation by pools of peptides derived from p24 and RT following various immunization protocols;

FIGS. 10-12 show the results of experiments discussed in Example 3, specifically:

FIG. 10 shows the lymphoproliferative response of rabbit PBMC against peptide pools covering the F4 sequence;

FIG. 11 shows the timecourse of antibody responses against F4;

FIGS. 12a and 12b shows antibody responses (on day 77) against F4 components p24 and RT respectively;

FIG. 13 shows the quantification of HIV-1-specific CD4 T cells;

FIG. 14 shows distribution of the frequency of F4-specific CD4 T cells 7 days after two immunizations;

FIG. 15 shows cytokine production of F4-specific CD4 T cells 7 days after two immunizations;

FIG. 16 shows quantification of HIV-1-specific CD8 T cells;

FIG. 17 shows cytokine production of F4-specific CD8 T cells 7 days after two immunizations;

FIG. 18 shows quantification of CSP-specific CD4 T cells;

FIG. 19 shows quantification of CSP-specific CD8 T cells;

FIG. 20 shows quantification of CSP(N-term)-specific CD4 T cells;

FIG. 21 shows quantification of CSP(C-term)-specific CD4 T cells;

FIG. 22 shows quantification of CSP(N-term)-specific CD8 T cells;

FIG. 23 shows quantification of CSP(C-term)-specific CD8 T cells;

FIG. 24 shows quantification of CSP-specific antibody titers.

### SUMMARY OF SEQUENCE LISTINGS

| | Sequence Identifier |
|---|---|
| Amino acid or polynucleotide description | (SEQ ID No) |
| HIV Gag-RT-Nef (“GRN”) (Clade B) (cDNA) | 1 |
| HIV Gag-RT-Nef (“GRN”) (Clade B) (amino | 2 |
| acid) | |
| HIV Gag-RT-integrase-Nef (“GRIN”) (Clade A) | 3 |
| (cDNA) | |
| HIV Gag-RT-integrase-Nef (“GRIN”) (Clade A) | 4 |
| (amino acid) | |
| HIV gp140 (Clade A) (cDNA) | 5 |
| HIV gp140 (Clade A) (amino acid) | 6 |
| HIV gp120 (Clade B) (cDNA) | 7 |
| HIV gp120 (Clade B) (amino acid) | 8 |
| TB antigens fusion protein M72 (cDNA) | 9 |
| TB antigens fusion protein M72 (amino acid) | 10 |
| P. falciparum CS protein-derived antigen | 11 |
| (cDNA) | |
| P. falciparum CS protein-derived antigen | 12 |
| (amino acid) | |
| P. falciparum CS protein-derived fusion protein | 13 |
| “RTS” (cDNA) | |
| P. falciparum CS protein-derived fusion protein | 14 |
| “RTS” (amino acid) | |
| HIV p24-RT-Nef-p17 (cDNA) | 15 |
| HIV p24-RT-Nef-p17 (amino acid) | 16 |

The above recited sequences may be employed as polypeptides or polynucleotides encoding polypeptides of use in exemplary aspects of the invention. Said polypeptides may consist of or comprise the above mentioned sequences. Initial Met residues are optional. N-terminal His residues (including His residues immediately following an initial Met, as in SEQ ID No 9) are optional or an N-terminal His tag of a different length may be employed (eg typically up to 6 His residues may be employed to facilitate isolation of the protein). Analogue proteins which have significant sequence identity eg greater than 80% eg greater than 90% eg greater than 95% eg greater than 99% sequence identity over the whole length of the reference sequence may be employed, especially when the analogue protein has a similar function and particularly when the analogue protein is similarly immunogenic. For example up to 20 eg up to 10 eg 1-5 substitutions (eg conservative substitutions) may be tolerated. Nucleic acids which differ from those recited above which encode the same proteins, or the aforementioned analogue proteins, may be employed. Sequence identity may be determined by conventional means eg using BLAST. In one specific variant of SEQ ID No 16 that may be mentioned, reside 398 is Ser and not Cys.

### DETAILED DESCRIPTION OF THE INVENTION

As used herein the term “concomitantly” means wherein the one or more immunogenic polypeptides, the one or more adenoviral vectors and the adjuvant are administered within a period of no more than 12 hours eg within a period of no more than 1 hour, typically on one occasion e.g. in the course of the same visit to the health professional, for example the one or more immunogenic polypeptides, the one or more adenoviral vectors and the adjuvant are administered sequentially or simultaneously.

As used herein, the term “epitope” refers to an immunogenic amino acid sequence. An epitope may refer to an a minimum amino acid sequence of typically 6-8 amino acids which minimum sequence is immunogenic when removed from its natural context, for example when transplanted into a heterologous polypeptide. An epitope may also refer to that portion of a protein which is immunogenic, where the polypeptide containing the epitope is referred to as the antigen (or sometimes “polypeptide antigen”). A polypeptide or antigen may contain one or more (eg 2 or 3 or more) distinct epitopes. The term “epitope” embraces B-cell and T-cell epitopes. The term “T-cell epitope” embraces CD4+ T-cell epitopes and CD8+ T-cell epitopes (sometimes also referred to as CTL epitopes).

The term “immunogenic polypeptide” refers to a polypeptide which is immunogenic, that is to say it is capable of eliciting an immune response in a mammal, and therefore contains one or more epitopes (eg T-cell and/or B-cell epitopes). Immunogenic polypeptides may contain one or more polypeptide antigens eg in an unnatural arrangement such as in a fusion protein.

Immunogenic polypeptides will typically be recombinant proteins produced eg by expression in a heterologous host such as a bacterial host, in yeast or in cultured mammalian cells.

The term “polypeptide derived from a pathogen” means a polypeptide which partially or wholly contains sequences (i.e. antigens) which occur naturally in pathogens or bear a high degree of sequence identity thereto (eg more than 95% identity over a stretch of at least 10 eg at least 20 amino acids).

Immunogenic polypeptides may contain one or more (eg 1, 2, 3 or 4) polypeptide antigens.

Unless otherwise specified, an “immune response” may be a cellular and/or a humoral response.

In one embodiment of the invention one or more of said one or more first immunogenic polypeptides is substantially the same as one or more of said one or more second immunogenic polypeptides. For example one of the at least one first immunogenic polypeptides and one of the at least one second immunogenic polypeptides may have an overall sequence identity of 90% or more eg 95% or more eg 98% or 99% or more over the length of one or other immunogenic polypeptides.

In another embodiment of the invention one or more of said one or more first immunogenic polypeptides contains at least one antigen which is substantially the same as an antigen contained in one or more of said one or more second immunogenic polypeptides. For example one of the at least one first immunogenic polypeptides and one of the at least one second immunogenic polypeptides may have an overall sequence identity of 90% or more eg 95% or more eg 98% or 99% or more over a stretch of 20 amino acids or more eg 40 amino acids or more eg 60 amino acids or more.

Suitably one or more first immunogenic polypeptides comprise at least one T cell epitope.

Suitably one or more second immunogenic polypeptides comprise at least one T cell epitope.

Suitably the one or more first immunogenic polypeptides comprise at least one B cell epitope.

Suitably the one or more second immunogenic polypeptides comprise at least one B cell epitope

In another embodiment of the invention one or more of said one or more first immunogenic polypeptides and one or more of said one or more second immunogenic polypeptides share one or more identical B-cell and/or T-cell epitopes. Suitably they share one or more identical amino acid sequences of length 10 amino acids or more eg 15 amino acids or more eg 25 amino acids or more.

In another embodiment of the invention, none of the one or more of said one or more first immunogenic polypeptides is substantially the same as or contains any antigen in common with one or more of said one or more second immunogenic polypeptides, for example they may have an overall sequence identity of less than 90% over a stretch of 20 amino acids or more eg 40 amino acids or more eg 60 amino acids or more.

Thus, they may not share any B-cell or T-cell epitopes. For example, they may note share any identical amino acid sequences of length 10 amino acids or more eg at 15 amino acids or more eg 25 amino acids or more.

In one specific embodiment of the invention a first immunogenic polypeptide and a second immunogenic polypeptide contain the same antigens in the same arrangement or in a different arrangement (eg in a different arrangement). By “different arrangement” is meant that they may be arranged in a different order and/or they may be divided. In another specific embodiment of the invention a first immunogenic polypeptide and a second immunogenic polypeptide are the same.

The composition according to the invention may contain one first immunogenic polypeptide as the only immunogenic polypeptide in the composition. Alternatively the composition according to the invention may contain more than one first immunogenic polypeptides eg 2 or 3 or 4 or more immunogenic polypeptides.

The composition according to the invention may comprise one adenoviral vector. Alternatively it may comprise more than one adenoviral vector eg 2 adenoviral vectors.

In compositions according to the invention a adenoviral vector may comprise a heterologous polynucleotide which encodes for one second immunogenic polypeptide or it may comprise more than one heterologous polynucleotide which together encode for more than one second immunogenic polypeptide under the control of more than one promoter.

As well as for prophylactic vaccination, the compositions of the invention may also be used in individuals that are already infected with pathogen, and result in improved immunological control of the established infection. This is of particular interest when the pathogen is HIV. In the case of HIV, this control is believed to be achieved by CD8-positive T cells that specifically recognize HIV-infected cells. Such CD8-positive T cell response is maintained by the presence of HIV-specific CD4-positive helper T cells. Therefore, the induction of both types of immune response is particularly useful, and can be achieved by combining different vaccine compositions. A combination of an adjuvanted protein and a recombinant adenovirus is of particular interest. The HIV-infected patients that will benefit from the above-described vaccination are either in the primary infection, latency or terminal phase of HIV infection at the time of vaccination. The patients may or may not undergo other therapeutic treatment interventions against pathogen (in the case of HIV—for example highly active antiretroviral therapy) at the time of vaccination.

### Antigens

Antigens of use according to the invention are derived from pathogens. Pathogens include viruses, bacteria, protozoa and other parasitic organisms harmful to mammals including man.

Suitable polypeptide antigens to be administered as polypeptide or polynucleotide encoding polypeptide according to the invention include antigens derived from HIV (eg HIV-1), human herpes viruses (such as gH, gL gM gB gC gK gE or gD or derivatives thereof or Immediate Early protein such as ICP27, ICP 47, ICP4, ICP36 from HSV1 or HSV2), cytomegalovirus, especially Human, (such as gB or derivatives thereof), Epstein Barr virus (such as gp350 or derivatives thereof), Varicella Zoster Virus (such as gpI, II, III and IE63), or from a hepatitis virus such as hepatitis B virus (for example Hepatitis B Surface antigen, PreS1, PreS2 and Surface env proteins, Hepatitis B core antigen or pol), hepatitis C virus (eg Core, E1, E2, P7, NS2, NS3, NS4A, NS4B, NS5A and B) and hepatitis E virus antigen, or from other viral pathogens, such as paramyxoviruses: Respiratory Syncytial virus (such as F and G proteins or derivatives thereof), or antigens from parainfluenza virus, measles virus, mumps virus, human papilloma viruses (for example HPV6, 11, 16, 18, eg L1, L2, E1, E2, E3, E4, E5, E6, E7), flaviviruses (e.g. Yellow Fever Virus, Dengue Virus, Tick-borne encephalitis virus, Japanese Encephalitis Virus) or Influenza virus (such as haemaggluttin, nucleoprotein, NA, or M proteins, or combinations thereof), or antigens derived from bacterial pathogens such as Neisseria spp, including N. gonorrhea and N. meningitidis, eg, transferrin-binding proteins, lactoferrin binding proteins, PilC, adhesins); S. pyogenes (for example M proteins or fragments thereof, C5A protease, S. agalactiae, S. mutans; H. ducreyi; Moraxella spp, including M catarrhalis, also known as Branhamella catarrhalis (for example high and low molecular weight adhesins and invasins); Bordetella spp, including B. pertussis (for example pertactin, pertussis toxin or derivatives thereof, filamentecus hemagglutinin, adenylate cyclase, fimbriae), B. parapertussis and B. bronchiseptica; Mycobacterium spp., including M. tuberculosis, M. bovis, M. leprae, M. avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli (for example shiga toxin-like toxin or derivatives thereof); Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp., including C. trachomatis, C. pneumoniae, C. psittaci; Leptospira spp., including L. interrogans; Treponema spp., including T. pallidum (for example the rare outer membrane proteins), T. denticola, T. hyodysenteriae; or derived from parasites such as Plasmodium spp., including P. falciparum and P. vivax; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; leishmania spp., including L. major; Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans.

Further bacterial antigens include antigens derived from Streptococcus spp, including S. pneumoniae (PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (WO 90/06951; WO 99/03884). Other bacterial antigens include antigens derived from Haemophilus spp., including H. influenzae type B (for example PRP and conjugates thereof), non typeable H. influenzae, for example OMP26, high molecular weight adhesins, P5, P6, protein D and lipoprotein D, and fimbrin and fimbrin derived peptides (U.S. Pat. No. 5,843,464) or multiple copy variants or fusion proteins thereof.

In particular, the methods or compositions of the present invention may be used to protect against or treat viral disorders such as those caused by Hepatitis B virus, Hepatitis C virus, Human papilloma virus, Human immunodeficiency virus (HIV), or Herpes simplex virus; bacterial diseases such as those caused by Mycobacterium tuberculosis (TB) or Chlamydia sp; and protozoal infections such as malaria.

It is to be recognised that these specific disease states, pathogens and antigens have been referred to by way of example only, and are not intended to be limiting upon the scope of the present invention.

### TB Antigens

The pathogen may, for example, be Mycobacterium tuberculosis.

Exemplary antigens derived from M. tuberculosis are for example alpha-crystallin (HspX), HBHA, Rv1753, Rv2386, Rv2707, Rv2557, Rv2558, RPFs: Rv0837c, Rv1884c, Rv2389c, Rv2450, Rv1009, aceA (Rv0467), ESAT6, Tb38-1, Ag85A, -B or -C, MPT 44, MPT59, MPT45, HSP10, HSP65, HSP70, HSP 75, HSP90, PPD 19 kDa [Rv3763], PPD, 38 kDa [Rv0934]), PstS1, (Rv0932), SodA (Rv3846), Rv2031c, 16 kDa, Ra12, TbH9, Ra35, Tb38-1, Erd 14, DPV, MTI, MSL, DPPD, mTCC1, mTCC2, hTCC1 (WO 99/51748) and hTCC2, and especially Mtb32a, Ra35, Ra12, DPV, MSL, MTI, Tb38-1, mTCC1, TbH9 (Mtb39a), hTCC1, mTCC2 and DPPD. Antigens derived from M. tuberculosis also include fusion proteins and variants thereof where at least two, or for example, three polypeptides of M. tuberculosis are fused into a larger protein. Such fusions may comprise or consist of Ra12-TbH9, Ra35, Erd14-DPV-MTI, DPV-MTI-MSL, Erd14-DPV-MTI-MSL-mTCC2, Erd14-DPV-MTI-MSL, DPV-MTI-MSL-mTCC2, TbH9-DPV-MTI (WO 99/51748), Ra12-Tbh9-Ra35-Ag85B and Ra12-Tbh9-Ra35-mTCC2. A particular Ra12-Tbh9-Ra35 sequence that may be mentioned is defined by SEQ ID No 6 of WO2006/117240 together with variants in which Ser 704 of that sequence is mutated to other than serine, eg to Ala, and derivatives thereof incorporating an N-terminal His tag of an appropriate length (eg SEQ ID No 2 or 4 of WO2006/117240). See also SEQ ID No 10 which is a sequence containing an optional starting M and an optional N-terminal His-His tag (positions 2 and 3) and in which the Ala mutated relative to the wild-type Ser is at position 706.

### Chlamydia Antigens

The pathogen may, for example, be a Chlamydia sp. eg C trachomatis.

Exemplary antigens derived from Chlamydia sp eg C trachomatis are selected from CT858, CT 089, CT875, MOMP, CT622, PmpD, PmpG and fragments thereof, SWIB and immunogenic fragments of any one thereof (such as PmpDpd and PmpGpd) and combinations thereof. Preferred combinations of antigens include CT858, CT089 and CT875. Specific sequences and combinations that may be employed are described in WO2006/104890.

### Plasmodium Antigens

The pathogen may, for example be a parasite that causes malaria such as a Plasmodium sp. eg P falciparum or P vivax.

For example, antigens derived from P falciparum include circumsporozoite protein (CS protein), PfEMP-1, Pfs 16 antigen, MSP-1, MSP-3, LSA-1, LSA-3, AMA-1 and TRAP. A particular hybrid antigen that may be mentioned is RTS. RTS is a hybrid protein comprising substantially all the C-terminal portion of the circumsporozoite (CS) protein of P. falciparum linked via four amino acids of the preS2 portion of Hepatitis B surface antigen to the surface (S) antigen of hepatitis B virus. When expressed in yeast RTS is produced as a lipoprotein particle, and when it is co-expressed with the S antigen from HBV it produces a mixed particle known as RTS,S The structure or RTS and RTS,S is disclosed in WO 93/10152. TRAP antigens are described in WO 90/01496. Other Plasmodium antigens include P. falciparum EBA, GLURP, RAP1, RAP2, Sequestrin, Pf332, STARP, SALSA, PfEXP1, Pfs25, Pfs28, PFS27125, Pfs48/45, Pfs230 and their analogues in other Plasmodium spp. One embodiment of the present invention is a composition comprising RTS,S or CS protein or a fragment thereof such as the CS portion of RTS, S in combination with one or more further malarial antigens which may be selected for example from the group consisting of MSP-1, MSP-3, AMA-1, Pfs 16, LSA-1 or LSA-3. Possible antigens from P vivax include circumsporozoite protein (CS protein) and Duffy antigen binding protein and immunogenic fragments thereof, such as PvRII (see eg WO02/12292).

Thus in one suitable embodiment of the invention, the first and second immunogenic polypeptides are selected from antigens derived from Plasmodium falciparum and/or Plasmodium vivax.

For example, the first and/or second immunogenic polypeptides are selected from antigens derived from Plasmodium falciparum and/or Plasmodium vivax are selected from RTS (eg as RTS,S), circumsporozoite (CS) protein, MSP-1, MSP-3, AMA-1, LSA-1, LSA-3 and immunogenic derivatives thereof or immunogenic fragments thereof.

One specific derivative that may be mentioned is the hybrid protein known as RTS, especially when presented in the form of a mixed particle known as RTS,S.

An exemplary RTS sequence is shown in SEQ ID No 14.

An exemplary P. falciparum CS protein-derived antigen is shown in SEQ ID No 12. This particular sequence corresponds to the CSP sequence of P. falciparum (3D7 strain), which also contains a 19 aa insertion coming from 7G8 strain (81-100).

In one specific embodiment of the invention, a first immunogenic polypeptide is RTS,S and a second immunogenic polypeptide is the CS protein from Plasmodium falciparum or an immunogenic fragment thereof.

### HPV Antigens

The pathogen may, for example, be a Human Papilloma Virus.

Thus antigens of use in the present invention may, for example, be derived from the Human Papilloma Virus (HPV) considered to be responsible for genital warts (HPV 6 or HPV 11 and others), and/or the HPV viruses responsible for cervical cancer (HPV16, HPV18, HPV33, HPV51, HPV56, HPV31, HPV45, HPV58, HPV52 and others). In one embodiment the forms of genital wart prophylactic, or therapeutic, compositions comprise L1 particles or capsomers, and fusion proteins comprising one or more antigens selected from the HPV proteins E1, E2, E5 E6, E7, L1, and L2. In one embodiment the forms of fusion protein are: L2E7 as disclosed in WO96/26277, and proteinD (1/3)-E7 disclosed in PCT/EP98/05285.

A preferred HPV cervical infection or cancer, prophylaxis or therapeutic composition may comprise HPV 16 or 18 antigens. For example, L1 or L2 antigen monomers, or L1 or L2 antigens presented together as a virus like particle (VLP) or the L1 alone protein presented alone in a VLP or capsomer structure. Such antigens, virus like particles and capsomer are per se known. See for example WO94/00152, WO94/20137, WO94/05792, and WO93/02184. Additional early proteins may be included alone or as fusion proteins such as E7, E2 or preferably E5 for example; particularly preferred embodiments of this includes a VLP comprising L1E7 fusion proteins (WO 96/11272). In one embodiment the HPV 16 antigens comprise the early proteins E6 or E7 in fusion with a protein D carrier to form Protein D-E6 or E7 fusions from HPV 16, or combinations thereof; or combinations of E6 or E7 with L2 (WO 96/26277). Alternatively the HPV 16 or 18 early proteins E6 and E7, may be presented in a single molecule, preferably a Protein D-E6/E7 fusion. Such a composition may optionally provide either or both E6 and E7 proteins from HPV 18, preferably in the form of a Protein D-E6 or Protein D-E7 fusion protein or Protein D E6/E7 fusion protein. Additionally antigens from other HPV strains, preferably from strains HPV 31 or 33 may be employed.

### HIV Antigens

The pathogen may, for example, be HIV eg HIV-1.

Thus, antigens may be selected from HIV derived antigens, particularly HIV-1 derived antigens.

HIV Tat and Nef proteins are early proteins, that is, they are expressed early in infection and in the absence of structural protein.

The Nef gene encodes an early accessory HIV protein which has been shown to possess several activities. For example, the Nef protein is known to cause the removal of CD4, the HIV receptor, from the cell surface, although the biological importance of this function is debated. Additionally Nef interacts with the signal pathway of T cells and induces an active state, which in turn may promote more efficient gene expression. Some HIV isolates have mutations or deletions in this region, which cause them not to encode functional protein and are severely compromised in their replication and pathogenesis in vivo.

The Gag gene is translated from the full-length RNA to yield a precursor polyprotein which is subsequently cleaved into 3-5 capsid proteins; the matrix protein p17, capsid protein p24 and nucleic acid binding protein (Fundamental Virology, Fields B N, Knipe D M and Howley M 1996 2. Fields Virology vol 2 1996).

The Gag gene gives rise to the 55-kilodalton (Kd) Gag precursor protein, also called p55, which is expressed from the unspliced viral mRNA. During translation, the N terminus of p55 is myristoylated, triggering its association with the cytoplasmic aspect of cell membranes. The membrane-associated Gag polyprotein recruits two copies of the viral genomic RNA along with other viral and cellular proteins that triggers the budding of the viral particle from the surface of an infected cell. After budding, p55 is cleaved by the virally encoded protease (a product of the Pol gene) during the process of viral maturation into four smaller proteins designated MA (matrix [p17]), CA (capsid [p24]), NC (nucleocapsid [p9]), and p6. (4).

In addition to the 3 major Gag proteins (p17, p24 and p9), all Gag precursors contain several other regions, which are cleaved out and remain in the virion as peptides of various sizes. These proteins have different roles e.g. the p2 protein has a proposed role in regulating activity of the protease and contributes to the correct timing of proteolytic processing.

The MA polypeptide is derived from the N-terminal, myristoylated end of p55. Most MA molecules remain attached to the inner surface of the virion lipid bilayer, stabilizing the particle. A subset of MA is recruited inside the deeper layers of the virion where it becomes part of the complex which escorts the viral DNA to the nucleus. These MA molecules facilitate the nuclear transport of the viral genome because a karyophilic signal on MA is recognized by the cellular nuclear import machinery. This phenomenon allows HIV to infect non-dividing cells, an unusual property for a retrovirus.

The p24 (CA) protein forms the conical core of viral particles. Cyclophilin A has been demonstrated to interact with the p24 region of p55 leading to its incorporation into HIV particles. The interaction between Gag and cyclophilin A is essential because the disruption of this interaction by cyclosporine inhibits viral replication.

The NC region of Gag is responsible for specifically recognizing the so-called packaging signal of HIV. The packaging signal consists of four stem loop structures located near the 5′ end of the viral RNA, and is sufficient to mediate the incorporation of a heterologous RNA into HIV-1 virions. NC binds to the packaging signal through interactions mediated by two zinc-finger motifs. NC also facilitates reverse transcription.

The p6 polypeptide region mediates interactions between p55 Gag and the accessory protein Vpr, leading to the incorporation of Vpr into assembling virions. The p6 region also contains a so-called late domain which is required for the efficient release of budding virions from an infected cell.

The Pol gene encodes three proteins having the activities needed by the virus in early infection, reverse transcriptase RT, protease, and the integrase protein needed for integration of viral DNA into cellular DNA. The primary product of Pol is cleaved by the virion protease to yield the amino terminal RT peptide which contains activities necessary for DNA synthesis (RNA and DNA directed DNA polymerase, ribonuclease H) and carboxy terminal integrase protein. HIV RT is a heterodimer of full-length RT (p66) and a cleavage product (p51) lacking the carboxy terminal RNase H domain.

RT is one of the most highly conserved proteins encoded by the retroviral genome. Two major activities of RT are the DNA Pol and ribonuclease H. The DNA Pol activity of RT uses RNA and DNA as templates interchangeably and like all DNA polymerases known is unable to initiate DNA synthesis de novo, but requires a pre existing molecule to serve as a primer (RNA).

The RNase H activity inherent in all RT proteins plays the essential role early in replication of removing the RNA genome as DNA synthesis proceeds. It selectively degrades the RNA from all RNA-DNA hybrid molecules. Structurally the polymerase and ribo H occupy separate, non-overlapping domains within the Pol covering the amino two thirds of the Pol.

The p66 catalytic subunit is folded into 5 distinct subdomains. The amino terminal 23 of these have the portion with RT activity. Carboxy terminal to these is the RNase H domain.

After infection of the host cell, the retroviral RNA genome is copied into linear double stranded DNA by the reverse transcriptase that is present in the infecting particle. The integrase (reviewed in Skalka AM '99 Adv in Virus Res 52 271-273) recognises the ends of the viral DNA, trims them and accompanies the viral DNA to a host chromosomal site to catalyse integration. Many sites in the host DNA can be targets for integration. Although the integrase is sufficient to catalyse integration in vitro, it is not the only protein associated with the viral DNA in vivo—the large protein—viral DNA complex isolated from the infected cells has been denoted the pre integration complex. This facilitates the acquisition of the host cell genes by progeny viral genomes.

The integrase is made up of 3 distinct domains, the N terminal domain, the catalytic core and the C terminal domain. The catalytic core domain contains all of the requirements for the chemistry of polynucleotidyl transfer.

HIV-1 derived antigens for us in the invention may thus for example be selected from Gag (for example full length Gag), p17 (a portion of Gag), p24 (another portion of Gag), p41, p40, Pol (for example full length Pol), RT (a portion of Pol), p51 (a portion of RT), integrase (a portion of Pol), protease (a portion of Pol), Env, gp120, gp140 or gp160, gp41, Nef, Vif, Vpr, Vpu, Rev, Tat and immunogenic derivatives thereof and immunogenic fragments thereof, particularly Env, Gag, Nef and Pol and immunogenic derivatives thereof and immunogenic fragments thereof including p17, p24, RT and integrase. HIV vaccines may comprise polypeptides and/or polynucleotides encoding polypeptides corresponding to multiple different HIV antigens for example 2 or 3 or 4 or more HIV antigens which may be selected from the above list. Several different antigens may, for example, be comprised in a single fusion protein. More than one first immunogenic polypeptide and/or more than one second immunogenic polypeptide each of which is an HIV antigen or a fusion of more than one antigen may be employed.

For example an antigen may comprise Gag or an immunogenic derivative or immunogenic fragment thereof, fused to RT or an immunogenic derivative or immunogenic fragment thereof, fused to Nef or an immunogenic derivative or immunogenic fragment thereof wherein the Gag portion of the fusion protein is present at the 5′ terminus end of the polypeptide.

A Gag sequence of use according to the invention may exclude the Gag p6 polypeptide encoding sequence. A particular example of a Gag sequence for use in the invention comprises p17 and/or p24 encoding sequences.

A RT sequence may contain a mutation to substantially inactivate any reverse transcriptase activity (see WO03/025003).

The RT gene is a component of the bigger pol gene in the HIV genome. It will be understood that the RT sequence employed according to the invention may be present in the context of Pol, or a fragment of Pol corresponding at least to RT. Such fragments of Pol retain major CTL epitopes of Pol. In one specific example, RT is included as just the p51 or just the p66 fragment of RT.

The RT component of the fusion protein or composition according to the invention optionally comprises a mutation to remove a site which serves as an internal initiation site in prokaryotic expression systems.

Optionally the Nef sequence for use in the invention is truncated to remove the sequence encoding the N terminal region i.e. removal of from 30 to 85 amino acids, for example from 60 to 85 amino acids, particularly the N terminal 65 amino acids (the latter truncation is referred to herein as trNef). Alternatively or additionally the Nef may be modified to remove the myristylation site. For example the Gly 2 myristylation site may be removed by deletion or substitution. Alternatively or additionally the Nef may be modified to alter the dileucine motif of Leu 174 and Leu 175 by deletion or substitution of one or both leucines. The importance of the dileucine motif in CD4 downregulation is described e.g. in Bresnahan P. A. et al (1998) Current Biology, 8(22): 1235-8.

The Env antigen may be present in its full length as gp160 or truncated as gp140 or shorter (optionally with a suitable mutation to destroy the cleavage site motif between gp120 and gp41). The Env antigen may also be present in its naturally occurring processed form as gp120 and gp41. These two derivatives of gp160 may be used individually or together as a combination. The aforementioned Env antigens may further exhibit deletions (in particular of variable loops) and truncations. Fragments of Env may be used as well.

An exemplary gp120 sequence is shown in SEQ ID No 8. An exemplary gp140 sequence is shown in SEQ ID No 6.

Immunogenic polypeptides according to the invention may comprise Gag, Pol, Env and Nef wherein at least 75%, or at least 90% or at least 95%, for example, 96% of the CTL epitopes of these native antigens are present.

In immunogenic polypeptides according to the invention which comprise p17/p24 Gag, p66 RT, and truncated Nef as defined above, 96% of the CT-L epitopes of the native Gag, Pol and Nef antigens are suitably present.

One embodiment of the invention provides an immunogenic polypeptide containing p17, p24 Gag, p66 RT, truncated Nef (devoid of nucleotides encoding terminal amino-acids 1-85—“trNef”) in the order Gag, RT, Nef. In polynucleotides encoding immunogenic polypeptides of the invention, suitably the P24 Gag and P66 RT are codon optimized.

Specific polynucleotide constructs and corresponding polypeptide antigens according to the invention include:

1. p17, p24 (codon optimised) Gag-p66 RT (codon optimised)-truncated Nef;
2. truncated Nef-p66 RT (codon optimised)-p17, p24 (codon optimised) Gag;
3. truncated Nef-p17, p24 (codon optimised) Gag-p66 RT (codon optimised);
4. p66 RT (codon optimised)-p17, p24 (codon optimised) Gag-truncated Nef;
5. p66 RT (codon optimised)-truncated Nef-p17, p24 (codon optimised) Gag;
6. p17, p24 (codon optimised) Gag-truncated Nef-p66 RT (codon optimised).

An exemplary fusion is a fusion of Gag, RT and Nef particularly in the order Gag-RT-Nef (see eg SEQ ID No 2). Another exemplary fusion is a fusion of p17, p24, RT and Nef particularly in the order p24-RT-Nef-p17 (see eg SEQ ID No 16, referred to elsewhere herein as “F4”).

In another embodiment an immunogenic polypeptide contains Gag, RT, integrase and Nef, especially in the order Gag-RT-integrase-Nef (see eg SEQ ID No 4).

In other embodiments the HIV antigen may be a fusion polypeptide which comprises Nef or an immunogenic derivative thereof or an immunogenic fragment thereof, and p17 Gag and/or p24 Gag or immunogenic derivatives thereof or immunogenic fragments thereof, wherein when both p17 and p24 Gag are present there is at least one HIV antigen or immunogenic fragment between them.

For example, Nef is suitably full length Nef.

For example p17 Gag and p24 Gag are suitably full length p17 and p24 respectively.

In one embodiment an immunogenic polypeptide comprises both p17 and p24 Gag or immunogenic fragments thereof. In such a construct the p24 Gag component and p17 Gag component are separated by at least one further HIV antigen or immunogenic fragment, such as Nef and/or RT or immunogenic derivatives thereof or immunogenic fragments thereof. See WO2006/013106 for further details.

In fusion proteins which comprise p24 and RT, it may be preferable that the p24 precedes the RT in the construct because when the antigens are expressed alone in E. coli better expression of p24 than of RT is observed.

Some constructs according to the invention include the following:

1. p24-RT-Nef-p17
2. p24-RT*-Nef-p17
3. p24-p51RT-Nef-p17

4. p24-p51RT*-Nef-p17

5. p17-p51RT-Nef

6. p17-p51RT*-Nef

### 7. Nef-p17

8. Nef-p17 with linker
9. p17-Nef
10. p17-Nef with linker * represents RT methionine₅₉₂ mutation to lysine

In another aspect the present invention provides a fusion protein of HIV antigens comprising at least four HIV antigens or immunogenic fragments, wherein the four antigens or fragments are or are derived from Nef, Pol and Gag. Preferably Gag is present as two separate components which are separated by at least one other antigen in the fusion. Preferably the Nef is full length Nef. Preferably the Pol is p66 or p51RT. Preferably the Gag is p17 and p24 Gag. Other preferred features and properties of the antigen components of the fusion in this aspect of the invention are as described herein.

Preferred embodiments of this aspect of the invention are the four component fusions as already listed above:

1. p24-RT-Nef-p17

2. p24-RT*-Nef-p17

3. p24-p51RT-Nef-p17

4. p24-p51RT*-Nef-p17

The immunogenic polypeptides of the present invention may have linker sequences present in between the sequences corresponding to particular antigens such as Gag, RT and Nef. Such linker sequences may be, for example, up to 20 amino acids in length. In a particular example they may be from 1 to 10 amino acids, or from 1 to 6 amino acids, for example 4-6 amino acids.

Further description of such suitable HIV antigens can be found in WO03/025003.

HIV antigens of the present invention may be derived from any HIV clade, for example lade A, clade B or clade C. For example the HIV antigens may be derived from clade A or B, especially B.

In one specific embodiment of the invention, a first immunogenic polypeptide is a polypeptide comprising Gag and/or Pol and/or Nef or a fragment or derivative of any of them (eg p24-RT-Nef-p17). In one specific embodiment of the invention a second immunogenic polypeptide is a polypeptide comprising Gap and/or Pol and/or Nef or a fragment or derivative of any of them (eg Gag-RT-Nef or Gag-RT-integrase-Nef).

Thus in one specific embodiment, a polypeptide comprising Gap and/or Pol and/or Nef or a fragment or derivative of any of them (eg p24-RT-Nef-p17) is a first immunogenic polypeptide and a polypeptide comprising Gap and/or Pol and/or Nef or a fragment or derivative of any of them (eg Gag-RT-Nef or Gag-RT-integrase-Nef) is a second immunogenic polypeptide.

In another specific embodiment of the invention, a first immunogenic polypeptide is Env or a fragment or derivative thereof eg gp120, gp140 or gp160 (especially gp120). In one specific embodiment of the invention a second immunogenic polypeptide is a polypeptide comprising Gag and/or Pol and/or Nef or a fragment or derivative of any of them (eg p24-RT-Nef-p17).

Thus in one specific embodiment, Env or a fragment or derivative thereof eg gp120, gp140 or gp160 (especially gp120) is a first immunogenic polypeptide and a polypeptide comprising Gag and/or Pol and/or Nef or a fragment or derivative of any of them (eg p24-RT-Nef-p17) is a second immunogenic polypeptide.

In another specific embodiment of the invention, a first immunogenic polypeptide is a polypeptide comprising Gag and/or Pol and/or Nef or a fragment or derivative of any of them (eg p24-RT-Nef-p17). In one specific embodiment of the invention a second immunogenic polypeptide is Env or a fragment or derivative thereof eg gp120, gp140 or gp160 (especially gp120).

Thus in one specific embodiment, a polypeptide comprising Gag and/or Pol and/or Nef or a fragment or derivative of any of them (eg p24-RT-Nef-p17) is a first immunogenic polypeptide and Env or a fragment or derivative thereof eg gp120, gp140 or gp160 (especially gp120) is a second immunogenic polypeptide.

### Immunogenic Derivatives and Immunogenic Fragments of Antigens

The aforementioned antigens may be employed in the form of immunogenic derivatives or immunogenic fragments thereof rather than the whole antigen.

As used herein the term “immunogenic derivative” in relation to an antigen of native origin refers to an antigen that have been modified in a limited way relative to its native counterparts. For example it may include a point mutation which may change the properties of the protein eg by improving expression in prokaryotic systems or by removing undesirable activity, eg enzymatic activity. Immunogenic derivatives will however be sufficiently similar to the native antigens such that they retain their antigenic properties and remain capable of raising an immune response against the native antigen. Whether or not a given derivative raises such an immune response may be measured by a suitably immunological assay such as an ELISA (for antibody responses) or flow cytometry using suitable staining for cellular markers (for cellular responses).

Immunogenic fragments are fragments which encode at least one epitope, for example a CTL epitope, typically a peptide of at least 8 amino acids. Fragments of at least 8, for example 8-10 amino acids or up to 20, 50, 60, 70, 100, 150 or 200 amino acids in length are considered to fall within the scope of the invention as long as the polypeptide demonstrates antigenicity, that is to say that the major epitopes (eg CTL epitopes) are retained by the polypeptide.

### Adenovirus

Adenoviral vectors of the present invention comprise one or more heterologous polynucleotides (DNA) which encode one or more immunogenic polypeptides.

Adenoviral vectors of use in the present invention may be derived from a range of mammalian hosts.

Adenoviruses (herein referred to as “Ad” or “Adv”) have a characteristic morphology with an icosohedral capsid consisting of three major proteins, hexon (II), penton base (III) and a knobbed fibre (IV), along with a number of other minor proteins, VI, VIII, IX, IIIa and IVa2 (Russell W. C. 2000, Gen Viriol, 81:2573-2604). The virus genome is a linear, double-stranded DNA with a terminal protein attached covalently to the 5′ termini, which have inverted terminal repeats (ITRs). The virus DNA is intimately associated with the highly basic protein VII and a small peptide termed mu. Another protein, V, is packaged with this DNA-protein complex and provides a structural link to the capsid via protein VI. The virus also contains a virus-encoded protease, which is necessary for processing of some of the structural proteins to produce mature infectious virus.

Over 100 distinct serotypes of adenovirus have been isolated which infect various mammalian species, 51 of which are of human origin. Thus one or more of the adenoviral vectors may be derived from a human adenovirus. Examples of such human-derived adenoviruses are Ad1, Ad2, Ad4, Ad5, Ad6, Ad11, Ad 24, Ad34, Ad35, particularly Ad5, Ad11 and Ad35. The human serotypes have been categorised into six subgenera (A-F) based on a number of biological, chemical, immunological and structural criteria.

Although Ad5-based vectors have been used extensively in a number of gene therapy trials, there may be limitations on the use of Ad5 and other group C adenoviral vectors due to preexisting immunity in the general population due to natural infection. Ad5 and other group C members tend to be among the most seroprevalent serotypes. Immunity to existing vectors may develop as a result of exposure to the vector during treatment. These types of preexisting or developed immunity to seroprevalent vectors may limit the effectiveness of gene therapy or vaccination efforts. Alternative adenovirus serotypes, thus constitute very important targets in the pursuit of gene delivery systems capable of evading the host immune response.

One such area of alternative serotypes are those derived from non human primates, especially chimpanzee adenoviruses. See U.S. Pat. No. 6,083,716 which describes the genome of two chimpanzee adenoviruses.

It has been shown that chimpanzee (“Pan” or “C”) adenoviral vectors induce strong immune responses to transgene products as efficiently as human adenoviral vectors (Fitzgerald et al. J. Immunol. 170:1416).

Non human primate adenoviruses can be isolated from the mesenteric lymph nodes of chimpanzees. Chimpanzee adenoviruses are sufficiently similar to human adenovirus subtype C to allow replication of E1 deleted virus in HEK 293 cells. Yet chimpanzee adenoviruses are phylogenetically distinct from the more common human serotypes (Ad2 and Ad5). Pan 6 is less closely related to and is serologically distinct from Pans 5, 7 and 9.

Thus one or more of the adenoviral vectors may be derived from a non-human primate adenovirus eg a chimpanzee adenovirus such as one selected from serotypes Pan5, Pan6, Pan7 and Pan9.

Adenoviral vectors may also be derived from more than one adenovirus serotype, and each serotype may be from the same or different source. For example they may be derived from more than one human serotype and/or more than one non-human primate serotype. Methods for constructing chimeric adenoviral vectors are disclosed in WO2005/001103.

There are certain size restrictions associated with inserting heterologous DNA into adenoviruses. Human adenoviruses have the ability to package up to 105% of the wild type genome length (Bett et al 1993, J Virol 67 (10), 5911-21). The lower packaging limit for human adenoviruses has been shown to be 75% of the wild type genome length (Parks et al 1995, J Virol 71(4), 3293-8).

One example of adenoviruses of use in the present invention are adenoviruses which are distinct from prevalent naturally occurring serotypes in the human population such as Ad2 and Ad5. This avoids the induction of potent immune responses against the vector which limits the efficacy of subsequent administrations of the same serotype by blocking vector uptake through neutralizing antibody and influencing toxicity.

Thus, the adenovirus may be an adenovirus which is not a prevalent naturally occurring human virus serotype. Adenoviruses isolated from animals have immunologically distinct capsid, hexon, penton and fibre components but are phylogenetically closely related. Specifically, the virus may be a non-human adenovirus, such as a simian adenovirus and in particular a chimpanzee adenovirus such as Pan 5, 6, 7 or 9. Examples of such strains are described in WO03/000283 and are available from the American Type Culture Collection, 10801 University Boulevard, Manassas, Va. 20110-2209, and other sources. Desirable chimpanzee adenovirus strains are Pan 5 [ATCC VR-591], Pan 6 [ATCC VR-592], and Pan 7 [ATCC VR-593].

Use of chimpanzee adenoviruses is thought to be advantageous over use of human adenovirus serotypes because of the lack of pre-existing immunity, in particular the lack of cross-neutralising antibodies, to adenoviruses in the target population. Cross-reaction of the chimpanzee adenoviruses with pre-existing neutralizing antibody responses is only present in 2% of the target population compared with 35% in the case of certain candidate human adenovirus vectors. The chimpanzee adenoviruses are distinct from the more common human subtypes Ad2 and Ad5, but are more closely related to human Ad4 of subgroup E, which is not a prevalent subtype. Pan 6 is less closely related to Pan 5, 7 and 9.

The adenovirus of the invention may be replication defective. This means that it has a reduced ability to replicate in non-complementing cells, compared to the wild type virus. This may be brought about by mutating the virus e.g. by deleting a gene involved in replication, for example deletion of the E1a, E1b, E3 or E4 gene.

The adenoviral vectors in accordance with the present invention may be derived from replication defective adenovirus comprising a functional E1 deletion. Thus the adenoviral vectors according to the invention may be replication defective due to the absence of the ability to express adenoviral E1a and E1b, i.e., are functionally deleted in E1a and E1b. The recombinant adenoviruses may also bear functional deletions in other genes [see WO 03/000283] for example, deletions in E3 or E4 genes. The adenovirus delayed early gene E3 may be eliminated from the adenovirus sequence which forms part of the recombinant virus. The function of E3 is not necessary to the production of the recombinant adenovirus particle. Thus, it is unnecessary to replace the function of this gene product in order to package a recombinant adenovirus useful in the invention. In one particular embodiment the recombinant adenoviruses have functionally deleted E1 and E3 genes. The construction of such vectors is described in Roy et al., Human Gene Therapy 15:519-530, 2004.

Recombinant adenoviruses may also be constructed having a functional deletion of the E4 gene, although it may be desirable to retain the E4 ORF6 function. Adenovirus vectors according to the invention may also contain a deletion in the delayed early gene E2a. Deletions may also be made in any of the late genes L1 through to L5 of the adenovirus genome. Similarly deletions in the intermediate genes IX and IVa may be useful.

Other deletions may be made in the other structural or non-structural adenovirus genes. The above deletions may be used individually, i.e. an adenovirus sequence for use in the present invention may contain deletions of E1 only. Alternatively, deletions of entire genes or portions thereof effective to destroy their biological activity may be used in any combination. For example in one exemplary vector, the adenovirus sequences may have deletions of the E1 genes and the E4 gene, or of the E1, E2a and E3 genes, or of the E1 and E3 genes (such as functional deletions in E1a and E1b, and a deletion of at least part of E3), or of the E1, E2a and E4 genes, with or without deletion of E3 and so on. Such deletions may be partial or full deletions of these genes and may be used in combination with other mutations, such as temperature sensitive mutations to achieve a desired result.

The adenoviral vectors can be produced on any suitable cell line in which the virus is capable of replication. In particular, complementing cell lines which provide the factors missing from the viral vector that result in its impaired replication characteristics (such as E1 and/or E4) can be used. Without limitation, such a cell line may be HeLa [ATCC Accession No. CCL 2], A549 [ATCC Accession No. CCL 185], HEK 293, KB [CCL 17], Detroit [e.g., Detroit 510, CCL 72] and WI-38 [CCL 75] cells, among others. These cell lines are all available from the American Type Culture Collection, 10801 University Boulevard, Manassas, Va. 20110-2209. Other suitable parent cell lines may be obtained from other sources, such as PER.C6© cells, as represented by the cells deposited under ECACC no. 96022940 at the European Collection of Animal Cell Cultures (ECACC) at the Centre for Applied Microbiology and Research (CAMR, UK) or Her 96 cells (Crucell).

The polynucleotide sequences which encode immunogenic polypeptides may be codon optimised for mammalian cells. Such codon-optimisation is described in detail in WO05/025614. Codon optimization for certain HIV sequences is further described in WO 03/025003.

In one embodiment of the present invention the polynucleotide constructs comprise an N-terminal leader sequence. The signal sequence, transmembrane domain and cytoplasmic domain are individually all optionally present or deleted. In one embodiment of the present invention all these regions are present but modified.

A promoter for use in the adenoviral vector according to the invention may be the promoter from HCMV IE gene, for example wherein the 5′ untranslated region of the HCMV IE gene comprising exon 1 is included and intron A is completely or partially excluded as described in WO 02/36792.

When several antigens are fused into a fusion protein, such protein would be encoded by a polynucleotide under the control of a single promoter.

In an alternative embodiment of the invention, several antigens may be expressed separately through individual promoters, each of said promoters may be the same or different. In yet another embodiment of the invention some of the antigens may form a fusion, linked to a first promoter and other antigen(s) may be linked to a second promoter, which may be the same or different from the first promoter.

Thus, the adenoviral vector may comprise one or more expression cassettes each of which encode one antigen under the control of one promoter. Alternatively or additionally it may comprise one or more expression cassettes each of which encode more than one antigen under the control of one promoter, which antigens are thereby expressed as a fusion. Each expression cassette may be present in more than one locus in the adenoviral vector.

The polynucleotide or polynucleotides encoding immunogenic polypeptides to be expressed may be inserted into any of the adenovirus deleted regions, for example into the E1 deleted region.

Although two or more polynucleotides encoding immunogenic polypeptides may be linked as a fusion, the resulting protein may be expressed as a fusion protein, or it may be expressed as separate protein products, or it may be expressed as a fusion protein and then subsequently broken down into smaller subunits.

### Adjuvant

Adjuvants are described in general in Vaccine Design—the Subunit and Adjuvant Approach eg Powell and Newman, Plenum Press, New York, 1995.

Suitable adjuvants include an aluminium salt such as aluminium hydroxide or aluminium phosphate, but may also be a salt of calcium, iron or zinc, or may be an insoluble suspension of acylated tyrosine, or acylated sugars, cationically or anionically derivatised polysaccharides, or polyphosphazenes.

In the formulation of the invention it is preferred that the adjuvant composition preferentially induces a Th1 response. However it will be understood that other responses, including other humoral responses, are not excluded.

It is known that certain vaccine adjuvants are particularly suited to the stimulation of either Th1 or Th2-type cytokine responses. Traditionally the best indicators of the Th1:Th2 balance of the immune response after a vaccination or infection includes direct measurement of the production of Th1 or Th2 cytokines by T lymphocytes in vitro after restimulation with antigen, and/or the measurement of the IgG1:IgG2a ratio of antigen specific antibody responses.

Thus, a Th1-type adjuvant is one which stimulates isolated T-cell populations to produce high levels of Th1-type cytokines in vivo (as measured in the serum) or ex vivo (cytokines that are measured when the cells are re-stimulated with antigen in vitro), and induces antigen specific immunoglobulin responses associated with Th1-type isotype.

Preferred Th1-type immunostimulants which may be formulated to produce adjuvants suitable for use in the present invention include and are not restricted to the following:

The Toll like receptor (TLR) 4 ligands, especially an agonist such as a lipid A derivative particularly monophosphoryl lipid A or more particularly 3 Deacylated monophoshoryl lipid A (3 D-MPL).

3 D-MPL is sold under the trademark MPL® by GlaxoSmithKline and primarily promotes CD4+ T cell responses characterized by the production of IFN-g (Th1 cells i.e. CD4 T helper cells with a type-1 phenotype). It can be produced according to the methods disclosed in GB 2 220 211 A. Chemically it is a mixture of 3-deacylated monophosphoryl lipid A with 3, 4, 5 or 6 acylated chains. Preferably in the compositions of the present invention small particle 3 D-MPL is used. Small particle 3 D-MPL has a particle size such that it may be sterile-filtered through a 0.22 μm filter. Such preparations are described in International Patent Application No. WO94/21292. Synthetic derivatives of lipid A are known and thought to be TLR δ agonists including, but not limited to:

OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO 95/14026)

OM 294 DP (3S,9R)-3-[(R)-dodecanoyloxytetradecanoylamino]-4-oxo-5-aza-9(R)-[(R)-3-hydroxytetradecanoylamino]decan-1,10-diol,1,10-bis(dihydrogenophosphate) (WO99/64301 and WO 00/0462)

OM 197 MP-Ac DP (3S-,9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127)

Other TLR4 ligands which may be used are alkyl Glucosaminide phosphates (AGPs) such as those disclosed in WO9850399 or U.S. Pat. No. 6,303,347 (processes for preparation of AGPs are also disclosed), or pharmaceutically acceptable salts of AGPs as disclosed in U.S. Pat. No. 6,764,840. Some AGPs are TLR4 agonists, and some are TLR4 antagonists. Both are thought to be useful as adjuvants.

Saponins are also preferred Th1 immunostimulants in accordance with the invention. Saponins are well known adjuvants and are taught in: Lacaille-Dubois, M and Wagner H. (1996. A review of the biological and pharmacological activities of saponins. Phytomedicine vol 2 pp 363-386). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit. Rev Ther Drug Carrier Syst, 1996, 12 (1-2):1-55; and EP 0 362 279 B1. The haemolytic saponins QS21 and QS17 (HPLC purified fractions of Quil A) have been described as potent systemic adjuvants, and the method of their production is disclosed in U.S. Pat. No. 5,057,540 and EP 0 362 279 B1. Also described in these references is the use of QS7 (a non-haemolytic fraction of Quil-A) which acts as a potent adjuvant for systemic vaccines. Use of QS21 is further described in Kensil et al. (1991. J. Immunology vol 146, 431-437). Combinations of QS21 and polysorbate or cyclodextrin are also known (WO 99/10008). Particulate adjuvant systems comprising fractions of QuilA, such as QS21 and QS7 are described in WO 96/33739 and WO 96/11711. One such system is known as an Iscom and may contain one or more saponins.

The adjuvant of the present invention may in particular comprises a Toll like receptor (TLR) 4 ligand, especially 3D-MPL, in combination with a saponin.

Other suitable adjuvants include TLR 9 ligands (agonists). Thus another preferred immunostimulant is an immunostimulatory oligonucleotide containing unmethylated CpG dinucleotides (“CpG”). CpG is an abbreviation for cytosine-guanosine dinucleotide motifs present in DNA. CpG is known in the art as being an adjuvant when administered by both systemic and mucosal routes (WO 96/02555, EP 468520, Davis et al., J. Immunol, 1998, 160(2):870-876; McCluskie and Davis, J. Immunol., 1998, 161(9):4463-6). Historically, it was observed that the DNA fraction of BCG could exert an anti-tumour effect. In further studies, synthetic oligonucleotides derived from BCG gene sequences were shown to be capable of inducing immunostimulatory effects (both in vitro and in vivo). The authors of these studies concluded that certain palindromic sequences, including a central CG motif, carried this activity. The central role of the CG motif in immunostimulation was later elucidated in a publication by Krieg, Nature 374, p546 1995. Detailed analysis has shown that the CG motif has to be in a certain sequence context, and that such sequences are common in bacterial DNA but are rare in vertebrate DNA. The immunostimulatory sequence is often: Purine, Purine, C, G, pyrimidine, pyrimidine; wherein the CG motif is not methylated, but other unmethylated CpG sequences are known to be immunostimulatory and may be used in the present invention.

In certain combinations of the six nucleotides a palindromic sequence is present. Several of these motifs, either as repeats of one motif or a combination of different motifs, can be present in the same oligonucleotide. The presence of one or more of these immunostimulatory sequences containing oligonucleotides can activate various immune subsets, including natural killer cells (which produce interferon γ and have cytolytic activity) and macrophages (Wooldrige et al Vol 89 (no. 8), 1977). Other unmethylated CpG containing sequences not having this consensus sequence have also now been shown to be immunomodulatory.

CpG when formulated into vaccines, is generally administered in free solution together with free antigen (WO 96/02555; McCluskie and Davis, supra) or covalently conjugated to an antigen (WO 98/16247), or formulated with a carrier such as aluminium hydroxide ((Hepatitis surface antigen) Davis et al. supra; Brazolot-Millan et al., Proc. Natl. Acad. Sci., USA, 1998, 95(26), 15553-8).

Other TLR9 agonists of potential interest include immunostimulatory CpR motif containing oligonucleotides and YpG motif containing oligonucleotides (Idera).

Such immunostimulants as described above may be formulated together with carriers, such as for example liposomes, oil in water emulsions, and or metallic salts, including aluminium salts (such as aluminium hydroxide). For example, 3D-MPL may be formulated with aluminium hydroxide (EP 0 689 454) or oil in water emulsions (WO 95/17210); QS21 may be advantageously formulated with cholesterol containing liposomes (WO 96/33739), oil in water emulsions (WO 95/17210) or alum (WO 98/15287); CpG may be formulated with alum (Davis et al. supra; Brazolot-Millan supra) or with other cationic carriers.

Combinations of immunostimulants are also preferred, in particular a combination of a monophosphoryl lipid A and a saponin derivative (WO 94/00153; WO 95/17210; WO 96/33739; WO 98/56414; WO 99/12565; WO 99/11241), more particularly the combination of QS21 and 3D-MPL as disclosed in WO 94/00153. Alternatively, a combination of CpG plus a saponin such as QS21 also forms a potent adjuvant for use in the present invention. Alternatively the saponin may be formulated in a liposome or in an Iscom and combined with an immunostimulatory oligonucleotide.

Thus, suitable adjuvant systems include, for example, a combination of monophosphoryl lipid A, preferably 3D-MPL, together with an aluminium salt (eg as described in WO00/23105).

An enhanced system involves the combination of a monophosphoryl lipid A and a saponin derivative particularly the combination of QS21 and 3D-MPL as disclosed in WO 94/00153, or a less reactogenic composition where the QS21 is quenched in cholesterol containing liposomes (DQ) as disclosed in WO 96/33739. This combination may additionally comprise an immunostimulatory oligonucleotide.

Thus an example adjuvant comprises QS21 and/or MPL and/or CpG.

A particularly potent adjuvant formulation involving QS21, 3D-MPL & tocopherol in an oil in water emulsion is described in WO 95/17210 and is another preferred formulation for use in the invention.

Another preferred formulation comprises a CpG oligonucleotide alone or together with an aluminium salt.

In a further aspect of the present invention there is provided a method of manufacture of a vaccine formulation as herein described, wherein the method comprises admixing one or more first immunogenic polypeptides according to the invention with a suitable adjuvant.

Particularly preferred adjuvants for use in the formulations according to the invention are as follows:

i) 3D-MPL+QS21 in a liposome (see eg Adjuvant B below)

### ii) Alum+3D-MPL

iii) Alum+QS21 in a liposome+3D-MPL

### iv) Alum+CpG

v) 3D-MPL+QS21+oil in water emulsion

### vi) CpG

vii) 3D-MPL+QS21 (eg in a liposome)+CpG
viii) QS21+CpG.

Preferably, the adjuvant is presented in the form of a liposome, ISCOM or an oil-in-water emulsion. In one example embodiment of the invention the adjuvant comprises an oil-in-water emulsion. In another example embodiment of the invention the adjuvant comprises liposomes.

Suitably the adjuvant component does not contain any virus. Thus suitably, compositions for use according to the invention do not contain any virus other than the one or more more adenoviral vectors comprising one or more heterologous polynucleotides encoding one or more second immunogenic polypeptides derived from a pathogen.

### Compositions, Dosage and Administration

In methods of the invention, the immunogenic polypeptide(s), the adenoviral vector(s) and the adjuvant are administered concomitantly.

Typically the adjuvant will be co-formulated with an immunogenic polypeptide. Suitably the adjuvant will also be co-formulated with any other immunogenic polypeptide to be administered.

Thus in one embodiment of the invention there is provided a method of raising an immune response which comprises administering (i) one or more first immunogenic polypeptides co-formulated with an adjuvant; and (ii) one or more adenoviral vectors comprising one or more heterologous polynucleotides encoding one or more second immunogenic polypeptides; wherein one or more first immunogenic polypeptides and adjuvant, and one or more adenoviral vectors are administered concomitantly.

By “co-formulated” is meant that the first immunogenic polypeptide and the adjuvant are contained within the same composition eg a pharmaceutical composition.

Typically the adenoviral vector is contained in a composition eg a pharmaceutical composition.

Alternatively, the one or more first immunogenic polypeptides, the one or more adenoviral vectors and an adjuvant are co-formulated.

Thus, there are provided compositions according to the invention which comprise one or more immunogenic polypeptides, one or more adenoviral vectors, and an adjuvant.

Compositions and methods according to the invention may involve use of more than one immunogenic polypeptide and/or more than one adenoviral vector. Use of multiple antigens is especially advantageous in raising protective immune responses to certain pathogens, such as HIV, M. tuberculosis and Plasmodium sp. Compositions according to the invention may comprise more than one adjuvant.

Compositions and methods employed according to the invention may typically comprise a carrier eg an aqueous buffered carrier. Protective components such as sugars may be included.

Compositions should be administered in sufficient amounts to transduce the target cells and to provide sufficient levels of gene transfer and expression and to permit pathogen-specific immune responses to develop thereby to provide a prophylactic or therapeutic benefit without undue adverse or with medically acceptable physiological effects, which can be determined by those skilled in the medical arts. Conventional and pharmaceutically acceptable routes of administration include, but are not limited to, direct delivery to the retina and other intraocular delivery methods, direct delivery to the liver, inhalation, intranasal, intravenous, intramuscular, intratracheal, subcutaneous, intradermal, epidermal, rectal, oral and other parenteral routes of administration. Routes of administration may be combined, if desired, or adjusted depending upon the gene product or the condition. The route of administration primarily will depend on the nature of the condition being treated. Most suitably the route is intramuscular, intradermal or epidermal.

Preferred tissues to target are muscle, skin and mucous membranes. Skin and mucous membranes are the physiological sites where most infectious antigens are normally encountered.

When the first immunogenic polypeptide, adjuvant and adenoviral vector are not co-formulated, the different formulations (eg polypeptide/adjuvant and adenoviral vector formulations) may be administered by the same route of administration or by different routes of administration.

Dosages of compositions in the methods will depend primarily on factors such as the condition being treated, the age, weight and health of the subject, and may thus vary among subjects. For example, a therapeutically effective adult human or veterinary dosage is generally in the range of from about 100 μL to about 100 mL of a carrier containing concentrations of from about 1×10⁶ to about 1×10¹⁵ particles, about 1×10¹¹ to 1×10¹³ particles, or about 1×10⁹ to 1×10¹² particles of virus together with around 1-1000 ug, or about 2-100 ug eg around 4-40 ug immunogenic polypeptide. Dosages will range depending upon the size of the animal and the route of administration. For example, a suitable human or veterinary dosage (for about an 80 kg animal) for intramuscular injection is in the range of about 1×10⁹ to about 5×10¹² virus particles and 4-40 ug protein per mL, for a single site. One of skill in the art may adjust these doses, depending on the route of administration, and the therapeutic or vaccinal application for which the composition is employed.

The amount of adjuvant will depend on the nature of the adjuvant and the immunogenic polypeptide, the condition being treated and the age, weight and health of the subject. Typically for human administration an amount of adjuvant of 1-100 ug eg 10-50 ug per dose may be suitable.

Suitably an adequate immune response is achieved by a single concomitant administration of the composition or compositions of the invention in methods of the invention.

However if the immune response is further enhanced by administration of a further dose of first immunogenic polypeptide, adjuvant and adenoviral vector on a second or subsequent occasion (for example after a month or two months) then such a protocol is embraced by the invention. We have found that good pathogen-specific CD4+ and/or CD8+ T-cell responses may typically be raised after a single concomitant administration of the composition or compositions of the invention in methods of the invention. However we have found that good pathogen-specific antibody responses may require a second or further concomitant administration of the composition or compositions of the invention.

The components of the invention may be combined or formulated with any suitable pharmaceutical excipient such as water, buffers and the like.

### EXAMPLES

### Adjuvant Preparations

### 1) The Preparation of Oil in Water Emulsion Followed the Protocol as Set Forth in WO 95/17210.

The emulsion contains: 42.72 mg/ml squalene, 47.44 mg/ml tocopherol, 19.4 mg/ml Tween 80.

The resulting oil droplets have a size of approximately 180 nm

Tween 80 was dissolved in phosphate buffered saline (PBS) to give a 2% solution in the PBS.

To provide 100 ml two fold concentrate, emulsion 5 g of DL alpha tocopherol and 5 ml of squalene were vortexed until mixed thoroughly. 90 ml of PBS/Tween solution was added and mixed thoroughly. The resulting emulsion was then passed through a syringe and finally microfluidised by using an M110S microfluidics machine. The resulting oil droplets have a size of approximately 180 nm

2) Preparation of Oil in Water Emulsion with QS21 and MPL

Sterile bulk emulsion was added to PBS to reach a final concentration of 500 μl of emulsion per ml (v/v). 3 D-MPL was then added. QS21 was then added Between each addition of component, the intermediate product was stirred for 5 minutes. Fifteen minutes later, the pH was checked and adjusted if necessary to 6.8+/−0.1 with NaOH or HCl. The final concentration of 3D-MPL and QS21 was 100 μg per ml for each.

### 3) Preparation of Liposomal MPL

A mixture of lipid (such as phosphatidylcholine either from egg-yolk or synthetic) and cholesterol and 3 D-MPL in organic solvent, was dried down under vacuum (or alternatively under a stream of inert gas). An aqueous solution (such as phosphate buffered saline) was then added, and the vessel agitated until all the lipid was in suspension. This suspension was then microfluidised until the liposome size was reduced to about 100 nm, and then sterile filtered through a 0.2 μm filter. Extrusion or sonication could replace this step.

Typically the cholesterol:phosphatidylcholine ratio was 1:4 (w/w), and the aqueous solution was added to give a final cholesterol concentration of 10 mg/ml.

The final concentration of MPL is 2 mg/ml.

The liposomes have a size of approximately 100 nm and are referred to as SUV (for small unilamelar vesicles). The liposomes by themselves are stable over time and have no fusogenic capacity.

### 4) Preparation of Adjuvant B (“Adj B”)

Sterile bulk of SUV was added to PBS. PBS composition was Na₂HPO₄: 9 mM; KH₂PO₄: 48 mM; NaCl: 100 mM pH 6.1. QS21 in aqueous solution was added to the SUV. The final concentration of 3D-MPL and QS21 was 100 μg per ml for each. This mixture is referred as Adjuvant B. Between each addition of component, the intermediate product was stirred for 5 minutes. The pH was checked and adjusted if necessary to 6.1+/−0.1 with NaOH or HCl.

Preparation of p24-RT-Nef-P17 Protein (“F4”)

F4 was prepared as described in WO2006/013106 Example 1, codon-optimised method.

### Preparation of Chimp Adenovirus Pan7 Containing Gag-RT-Nef Transgene (“Pan7GRN”)

### Construction of Gag, RT, Nef Plasmid.

Plasmid p73i-Tgrn

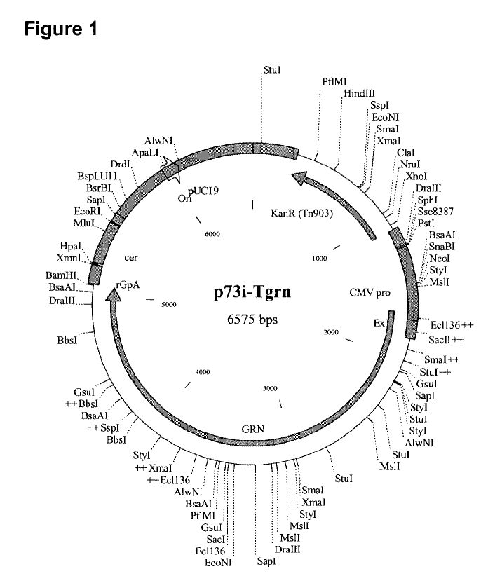

The full sequence of the Tgrn plasmid insert is given in SEQ ID No 1 and the plasmid construction shown graphically in FIG. 1. This contains p17 p24 (codon optimised) Gag, p66 RT (codon optimised and inactivated) and truncated Nef.

The plasmid P73i-Tgrn was prepared as described in WO03/025003 Examples 1-13.

Construction of the E1/E3 Deleted Pan 7 Adenovirus The E1/E3 deleted Pan 7 Adenovirus was prepared as described in WO2006/120034 Example 1.

Other serotypes of vectors can be constructed in a similar way. A full description of the construction of E1, E3 and E4 deletions in this and other Pan Adenovirus serotypes is given in WO03/0046124. Further information is also available in Human Gene Therapy 15:519-530.

Insertion of Gag, RT, Nef Sequence into Adenovirus

Using plasmid P73i-Tgrn, the GRN expression cassette was inserted into E1/E3 deleted Pan 7 adenovirus to produce C7-GRNc as described in WO2006/120034 Example 3. C7-GRNc is the Pan7GRN adenovirus component used in the examples set out herein.

### Example 1

### Immunogenicity Study in Mice Immunised with Adenovirus Component (Pan7GRN) and Protein Component (F4/Adjuvant B) Separately or with Both Adenovirus and Protein Components Co-Formulated Together

The mouse strain used was CB6F1 and 3 mice were used per timepoint. For immunisation with F4/adjuvant B (P), 1/10 of the human dose was injected i.e. 9 ug of F4 protein in 50 uL of adjuvant B. For immunisation with Pan7GRN (A), 10×10⁸ virus particles in 50 uL of saline (0.9% NaCl water for injection solution) was used. The Pan7GRN chimp adenovirus carries the genes coding for Gag (G), RT (R) and Nef (N).

The vaccination schedule was as follows:

| Group | Day 0 | Day 21 | Day 42 | Day 63 |
|---|---|---|---|---|
| 1 | — | — | F4/adj B | F4/adj B |
| 2 | | | Pan7GRN | Pan7GRN |
| 3 | F4/adj B | F4/adj B | Pan7GRN | Pan7GRN |
| 4 | Pan7GRN | Pan7GRN | F4/adj B | F4/adj B |
| 5 | — | — | — | F4/adj B/ |
| | | | | Pan7GRN |
| 6 | — | — | F4/adj B/ | F4/adj B/ |
| | | | Pan7GRN | Pan7GRN |
| 7 | — | — | adj B | adj B |
| 8 | — | — | — | — |

Thus it can be seen that in groups 1 and 2, the mice were immunized with 2 injections of protein (PP) or adenovirus (AA), respectively. Mice from groups 3 and 4, received a conventional prime-boost schedule: protein then adenovirus (PPAA) or the other way round (AAPP) whereas in groups 5 and 6, the mice received one or two injections of a combination (combo) of protein and adenovirus together according to the invention. Mice from group 7 only received adjuvant control whereas mice from group 6 were naive.

The following read-outs were performed:

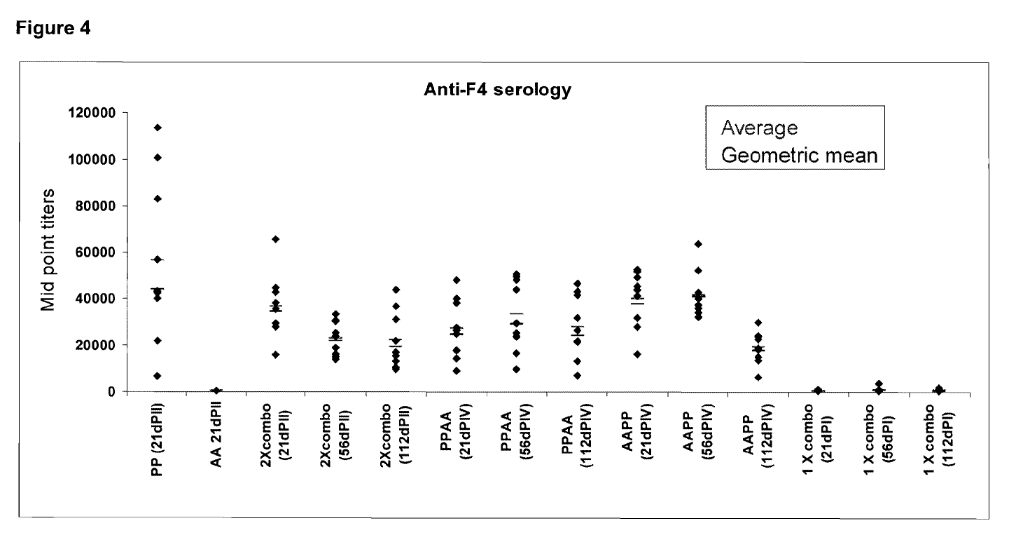

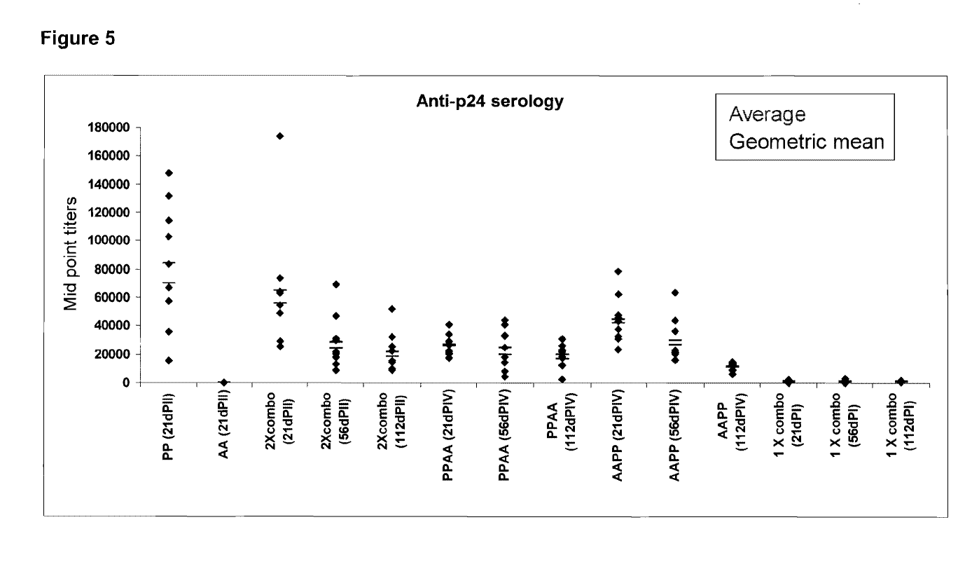

Antibody responses (ELISA performed on the sera from each individual animals from each group):
antibody response against F4 (FIG. 4)
antibody response against F4 components p24, RT, Nef and p17 (FIG. 5-8)

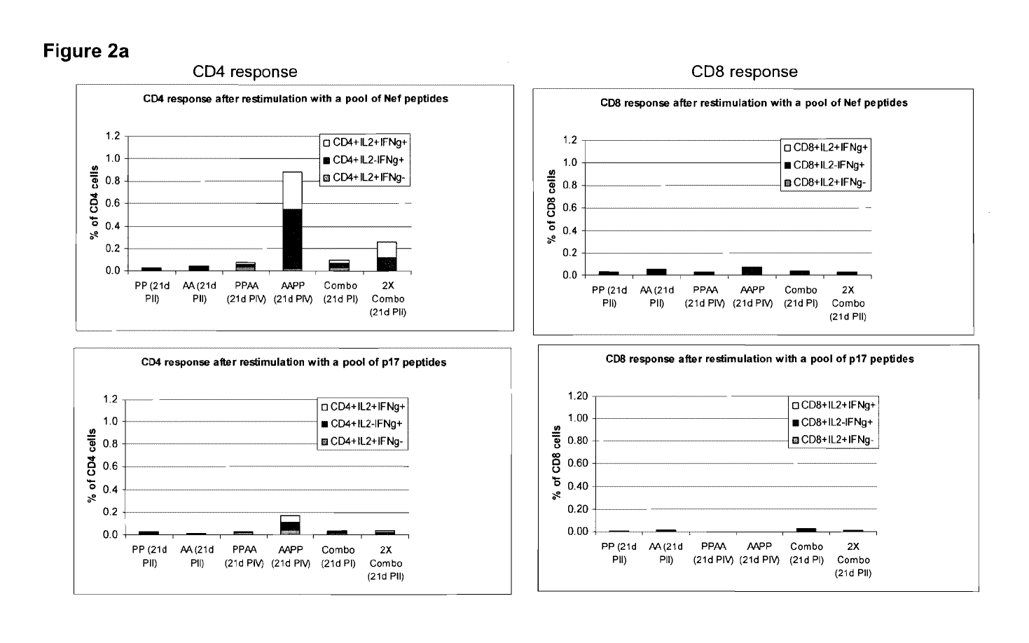

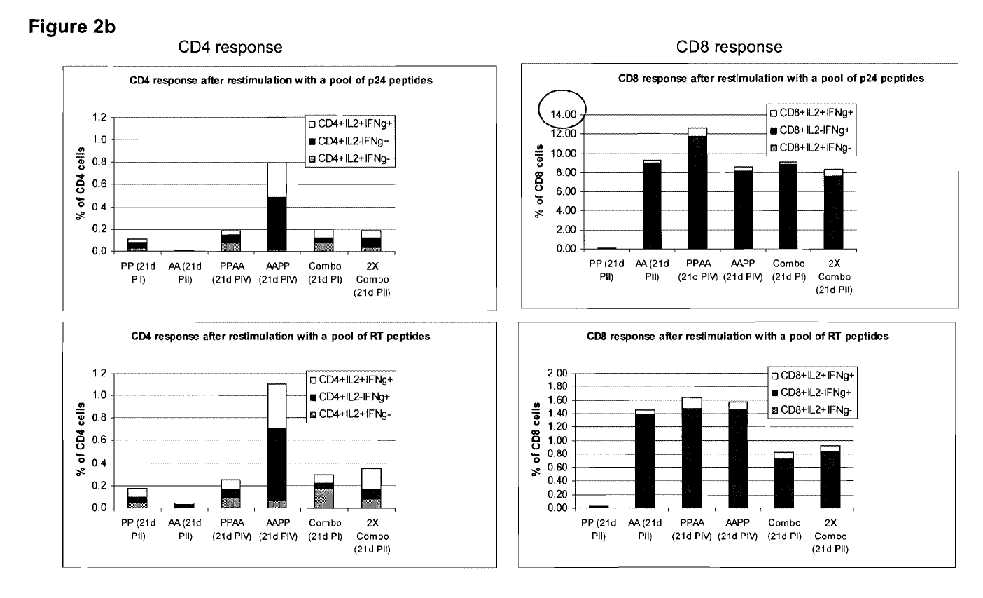

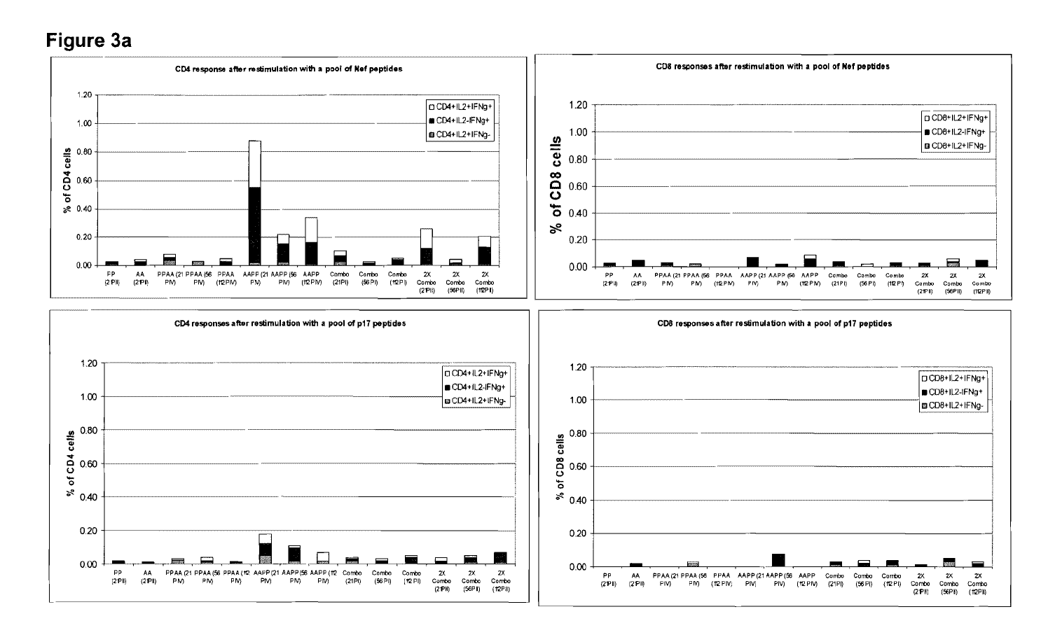

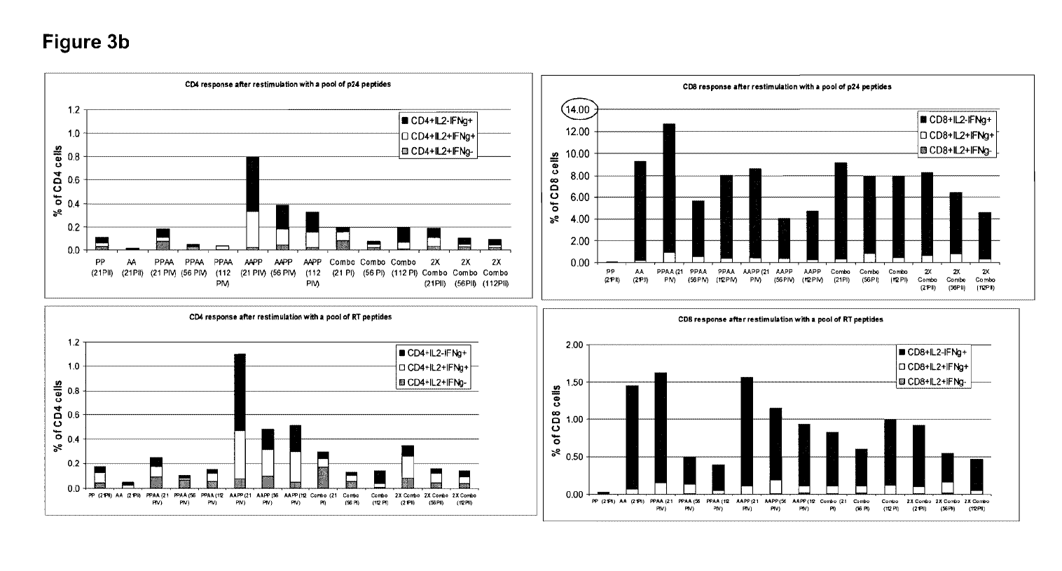

### Cellular Responses (FIGS. 2-3):

measured by flow cytometry following surface and intracellular cytokine staining after overnight restimulation of spleen cells with pools of peptides of p24, RT, Nef or p17. The spleen cells of 3 mice per timepoint and per group were pooled for the analysis.

For groups 1 and 2, samples were taken for measurement 21 days after the corresponding final immunisation. For the remaining groups, measurements were taken 21 days, 56 days and 112 days after the corresponding final immunisation.

### Results:

The results are shown in FIGS. 2-8.

The X axis labels correspond as follows:

PP—Group 1 animals following second immunisation
AA—Group 2 animals following second immunisation
PPAA—Group 3 animals following fourth immunisation
AAPP—Group 4 animals following fourth immunisation
Combo—Group 5 animals following immunisation
Combo×2—Group 6 animals following second immunisation

The measurement timepoints (21, 56 or 112 days post last immunisation) are indicated in parentheses.

### Cellular Responses (FIG. 2-3):

At the timepoints analysed, the data show that CD4+ T-cell responses were observed mainly against p24, RT and Nef.

As shown in FIGS. 2a and 2b (left panels), 21 days post last immunisation, the highest CD4+ T-cell responses are observed with two immunisations of adenovirus followed by two immunisations of protein/adjuvant (Group 4 animals). One injection of the combination of adenovirus/protein/adjuvant induces higher CD4+ T-cell levels than two injections of protein/adjuvant following restimulation with p24, RT or Nef peptides.

For restimulation by RT and Nef, two immunisations with the combination of adenovirus/protein/adjuvant induces a CD4+ T-cell response slightly higher than with one immunisation with the combination, whereas the responses with one or two immunisations were identical for p24.

At the timepoints analysed, the CD8+ T-cell responses are mainly observed against the p24 and RT peptides, and no significant numbers of CD8+ T-cells specific for Nef or p17 were detected.

As shown in FIGS. 2a and 2b (right panels), 21 days post last immunisation CD8+ T-cell responses were similar after one or two immunisations with the combination of adenovirus/protein/adjuvant. The CD8 response against p24 observed in groups immunised either (i) twice with adenovirus or (ii) twice with adenovirus followed by twice with protein or (iii) once or twice with the combination of adenovirus/protein/adjuvant were comparable to each other and slightly lower than the one from the group immunised twice with protein followed by twice with adenovirus. The CD8 response against RT observed in groups immunised once or twice with the combination of adenovirus/protein/adjuvant were comparable and slightly lower to the one from the groups immunised either (i) twice with adenovirus or (ii) twice with adenovirus followed by twice with protein or (iii) twice with protein followed by twice with adenovirus.

The CD4 and CD8 T cell responses were also analysed at later timepoints (56 and 112 days post last immunisation), when persistence of the responses can be determined (FIGS. 3a and 3b). The CD4 responses (FIGS. 3a and 3b, left panels) are mainly observed against p24, RT and Nef. At these timepoints, the highest CD4 responses are observed in the animals immunised twice with adenovirus followed by twice with protein. The CD4 response in mice immunised once or twice with the combination of adenovirus/protein/adjuvant were comparable to each other and generally higher than the response observed in groups immunised twice with protein followed by twice with adenovirus.

At the later timepoints, the CD8 response against p24 is the highest in the group immunised once with the combination of adenovirus/protein/adjuvant (FIG. 3b, right panel). It is comparable to the one from animals immunised twice with protein followed by twice with adenovirus and slightly higher than the one from the animals immunised either (i) twice with the combination of adenovirus/protein/adjuvant or (ii) twice with adenovirus followed by twice with protein. The latter two are comparable between each other. The CD8 response against RT is the highest and similar in groups immunised (i) twice with the combination of adenovirus/protein/adjuvant or (ii) twice with adenovirus followed by twice with protein. The CD8 response against RT from groups immunised (i) twice with the combination of adenovirus/protein/adjuvant or (ii) twice with protein followed by twice with adenovirus was slightly lower but comparable between each other (FIG. 3). As shown in FIG. 3a (right panel), no significant numbers of CD8+ T-cells specific for Nef or p17 were detected.

### Antibody Responses:

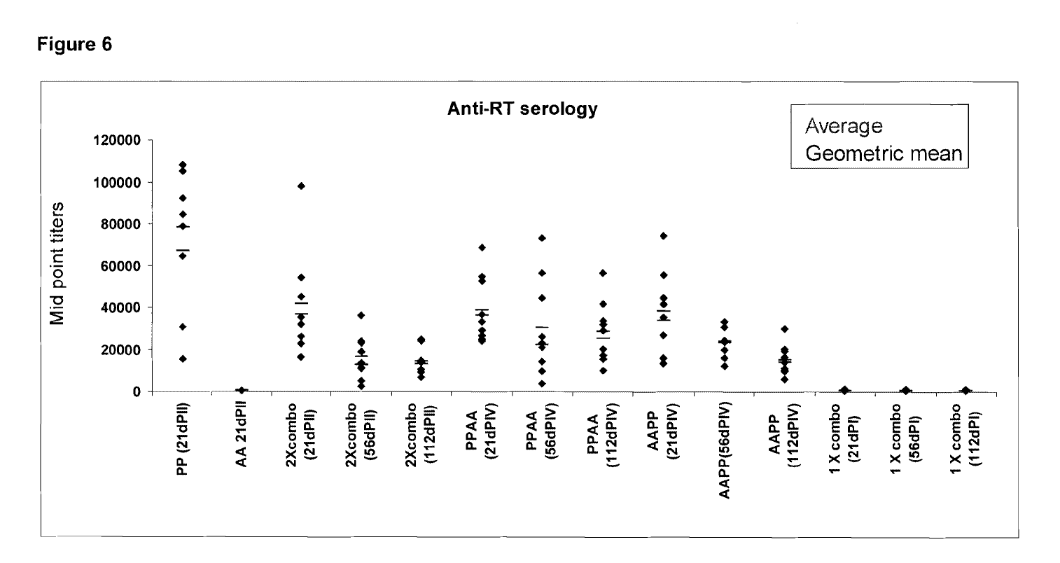

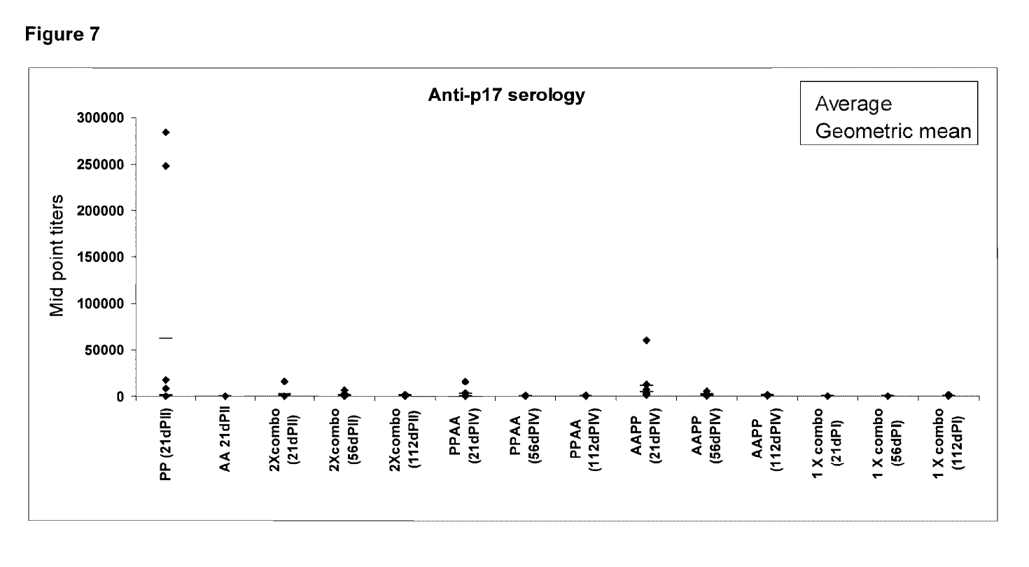

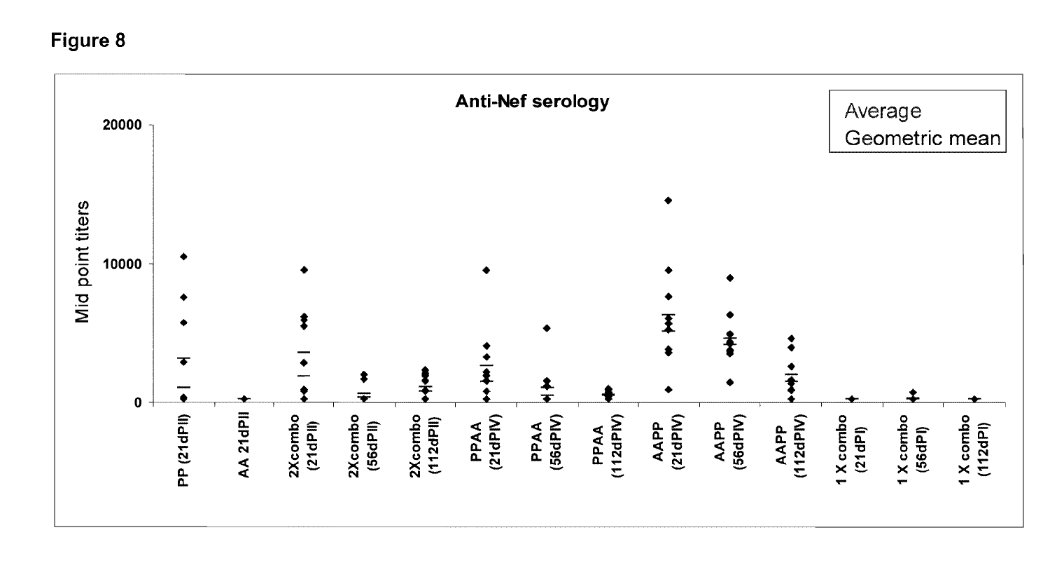

As shown in FIGS. 4 to 8, the antibody responses detected are mainly directed against p24 (FIG. 5), RT (FIG. 6) and Nef (FIG. 8). The anti-F4 (FIG. 4) response generally mimics the response observed against each of the p24, RT or Nef components and can be characterized as follows:
Low to no antibody response is detected in groups immunised (i) twice with adenovirus or (ii) once with the combination of adenovirus/protein/adjuvant;
The highest antibody responses usually detected in group immunised twice with the protein at 21 days post immunisation. However, it is also in this group that the highest variability between individuals is observed. In addition, for the anti-Nef serology, the group immunised twice with adenovirus followed by twice with protein appears to display the highest response, when compared to the other groups;
The response observed in groups immunised (i)) twice with the combination of adenovirus/protein/adjuvant or (ii) twice with protein followed by twice with adenovirus or (iii) twice with adenovirus followed by twice with protein are comparable, peak at 21 days post last immunisation and then slightly decrease over time.
   Antibody responses against p17 (FIG. 7) were very low to undetectable in all groups.

### Conclusion:

Globally, the highest antigen-specific cell-mediated immune response is observed in the AAPP treatment group after 4 immunisations. However, when comparing groups after 2 immunisations (i.e. AA, PP and 2× combo groups), the induction of both antigen-specific CD4 and CD8 T cell responses is only observed in the group immunised twice with the protein/adenovirus/adjuvant combination. In addition, similar levels of CD4 and CD8 T cell responses can be reached after a single injection of the protein/adenovirus/adjuvant combination. Moreover, in terms of persistence, the antigen-specific T cell responses observed 112 days after the 2^{nd} immunisation with the protein/adenovirus/adjuvant combination are comparable to the ones observed 112 days after the 4^{th} immunisations in the AAPP treatment group. Finally, it appears that 2 immunisations with the protein/adenovirus/adjuvant combination are needed to obtain an antibody response comparable to the one obtained in the group immunised twice with the adjuvanted protein, group that provided the highest antibody responses in general.

### Example 2

### Immunogenicity Study in Mice Immunised with Pan7GRN Adenovirus and F4 Protein/Adjuvant B Co-Formulated Together

The mouse strain used was CB6F1 with 9 mice per group. Mice were immunized once with a co-formulation of the F4 protein (1/10 of the human dose was injected i.e. 9 ug) together with 10×10⁸ virus particles of Pan7GRN, in 50 uL of adjuvant B or a dilution of the latter (1/2, 1/4 or 1/10). The CD4 and CD8 cellular responses against a pool of either Nef, p17, p24 or RT peptides were determined 21 days post immunization (3 pools of 3 spleens for each group).

The following read-out was performed:

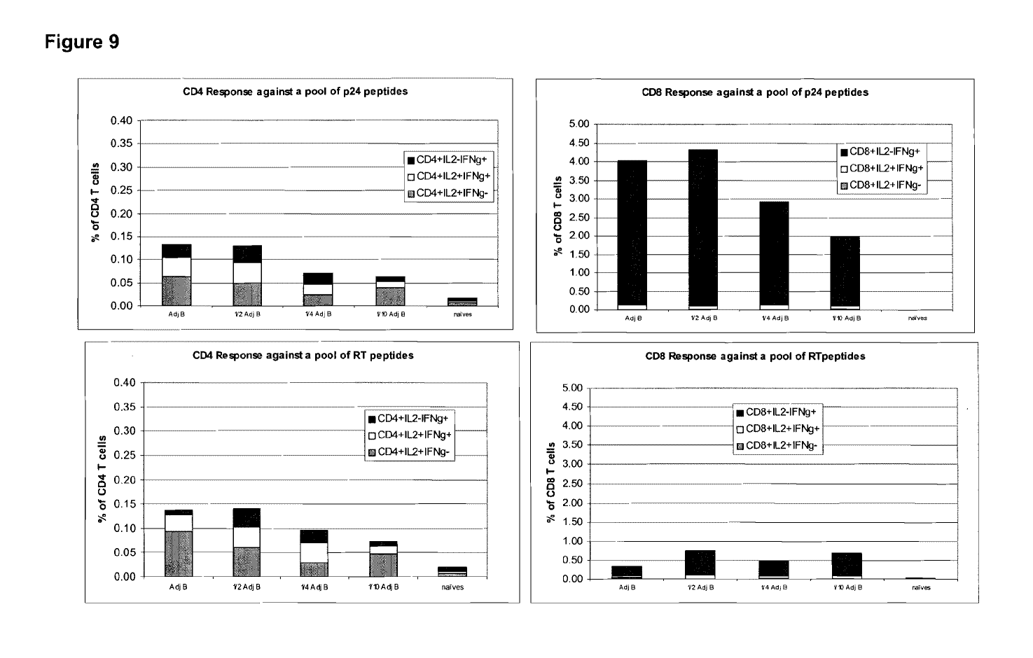

### Cellular Responses (FIG. 9):

measured by flow cytometry following surface and intracellular cytokine staining after overnight restimulation of spleen cells with pools of peptides of p24, RT, Nef or p17. The spleen cells were pooled (3 pools of 3 spleens per group) for the analysis.

### Results:

The results shown in FIG. 9 represent the cellular responses observed after restimulation with a pool of p24 or RT peptides.

The X axis labels correspond as follows:

Adj B—Mice immunised with 9 μgF4/10⁸vpPan7GRN/non-diluted adjuvant B
1/2 Adj B—Mice immunised with 9 μgF4/10⁸vpPan7GRN/adjuvant B diluted 1/2
1/4 Adj B—Mice immunised with 9 μgF4/10⁸vpPan7GRN/adjuvant B diluted 1/4
1/10 Adj B—Mice immunised with 9 μgF4/10⁸vpPan7GRN/adjuvant B diluted 1/10
Naïve—Naïve Mice (No Immunisation)

The results indicate that CD4 (FIG. 9, left panel) and CD8 (FIG. 9, right panel) responses are mainly observed against p24 and RT, with the CD8 T cell response specific to RT being lower than the one specific to p24. In addition, the results indicate that the CD4 responses against p24 and RT at 21 days post-immunisations in the groups immunised with the non-diluted adjuvant B or a 1/2 dilution of it are similar. These CD4 responses tend to decrease when the adjuvant is diluted 1/4. When the adjuvant B is diluted at 1/10, the CD4 responses observed are similar to the ones from groups immunised with the 1/4 dilution of the adjuvant B. The anti-CD8 responses against p24 are comparable whether the adjuvant is diluted 1/2 or not. However, the response decreases when the adjuvant B is diluted 1/4 and even more so if it is diluted 1/10. In contrast, such trends are not seen for the anti-RT CD8 responses where there is not a real dose range effect of the dose of adjuvant used.

### Conclusion:

CD4+ cells and CD8+ cells against F4 components were induced by a single administration of a composition containing an immunogenic polypeptide, an adenoviral vector containing a heterologous polynucleotide encoding an immunogenic polypeptide and an adjuvant, even when the latter was diluted. The impact of adjuvant dilution differed depending on the antigen-specific CD4 or CD8 responses of interest. In particular the highest responses observed were against p24 and the anti-p24 CD4 and CD8 T cell responses show a dose range effect correlating with the dose of adjuvant used in the combination vaccine. While the same effect can be observed for the anti-RT CD4 T cell response, the dose range effect of the dose of adjuvant used in the combo is less clear for the anti-RT CD8 T cell response. Finally, if we consider the global antigen-specific CD4 and CD8 T cell responses and sum the responses against the 4 antigens, a dose range can be observed.

### Example 3

### Immunogenicity Study in New Zealand White Rabbits Immunised with Pan7GRN or F4/Adjuvant B Sequentially or with Both Adenovirus and Protein Components Co-Formulated Together

For immunisation with F4/adjuvant B, the human dose was injected i.e. 90 ug of F4 protein in 500 uL of adjuvant B. For immunisation with Pan7GRN, 10×10¹⁰ or 10×10¹² virus particles in 500 uL of saline were used. For the immunization with both adenovirus and protein components co-formulated together, 90 ug of F4 protein, 10×10¹¹ virus particles of Pan7 GRN in 500 uL of adjuvant B were used.

The vaccination schedule was as follows:

| Group | Day 0 | Day 14 | Day 126 |
|---|---|---|---|
| 1 | F4/adj B | F4/adj B | F4/adj B |
| 2 | Pan7GRN 10\hat{ }10 | | Pan7GRN 10\hat{ }10 |
| 3 | Pan7GRN 10\hat{ }12 | | Pan7GRN 10\hat{ }12 |
| 4 | F4/adj B/ | F4/adj B/ | F4/adj B/ |
| | Pan7GRN 10\hat{ }11 | Pan7GRN 10\hat{ }11 | Pan7GRN 10\hat{ }11 |

There were 3 rabbits per group except for group 1 which included only 2 rabbits.

The following read-outs were performed:

Antibody Responses (ELISA Performed on the Sera from Each Individual Animals from Each Group):
antibody response against F4
antibody response against F4 components p24, RT, Nef and p17

### Lymphoproliferative Responses:

The lymphoproliferation was determined by the uptake of tritiated thymidine by peripheral blood mononuclear cells (isolated from whole blood after a density gradient) restimulated in vitro with pools of Nef, p17, p24 and/or RT peptides for 88 hours in the presence of tritiated thymidine for the last 16 hours of the incubation.

### Results:

### Lymphoproliferative Response:

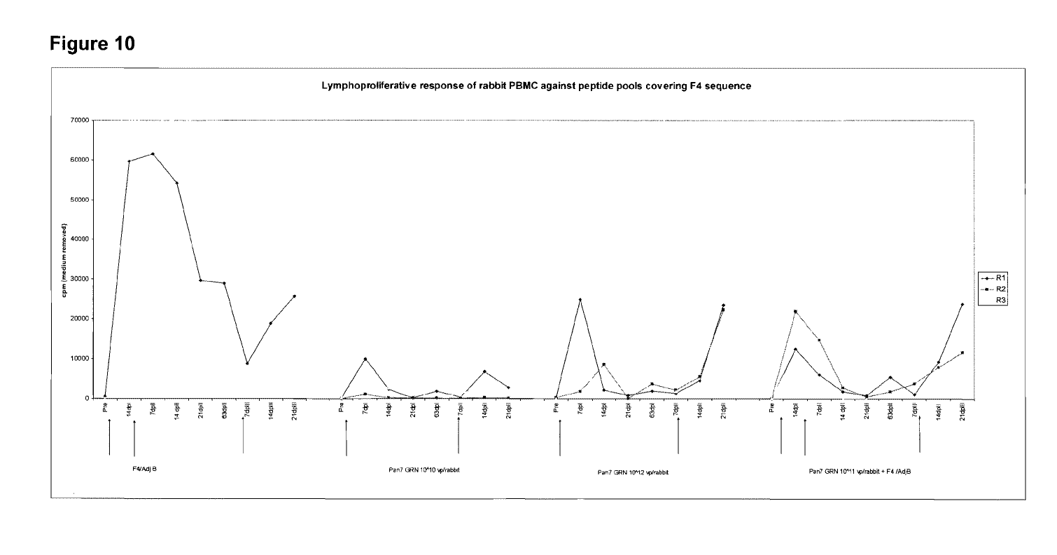

As shown in FIG. 10, the highest lymphoproliferative responses are observed in the group immunised twice with protein. The lymphoproliferative response from animals immunised twice with the combination of adenovirus/protein/adjuvant was observed in all rabbits from the group. It actually peaked after one injection and could be further recalled (at similar levels than after the 1^{st} injection) following a third injection of the combination of adenovirus/protein/adjuvant, suggesting that the first two injections did not induce a neutralizing response that would inhibit any response to a further similar injection. In its intensity, the proliferative response observed in rabbits immunised with the combination of adenovirus/protein/adjuvant was comparable to the one observed in animals immunised once or twice with 10¹² viral particles of adenovirus and appeared higher than the one from animals immunised once or twice with 10¹⁰ viral particles of adenovirus. Altogether, this suggests that using the combination of adenovirus/protein/adjuvant could decrease the dose of adenovirus to be used. Finally, after a third injection of the combination of adenovirus/protein/adjuvant, the response observed in group 4 was similar to the one from animals immunised 3 times with the protein (group 1).

### Serology:

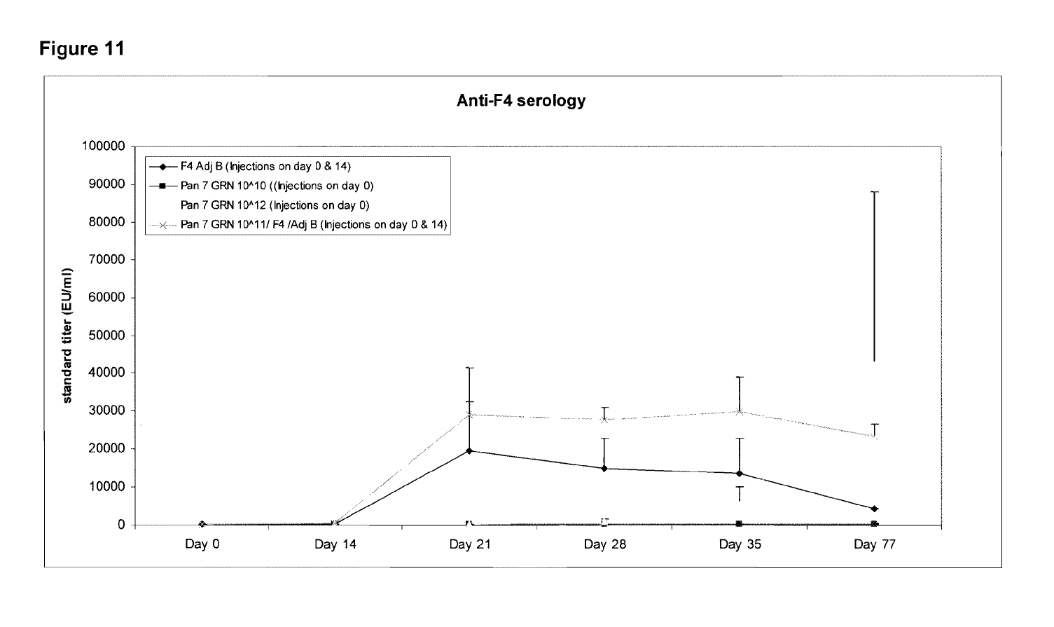

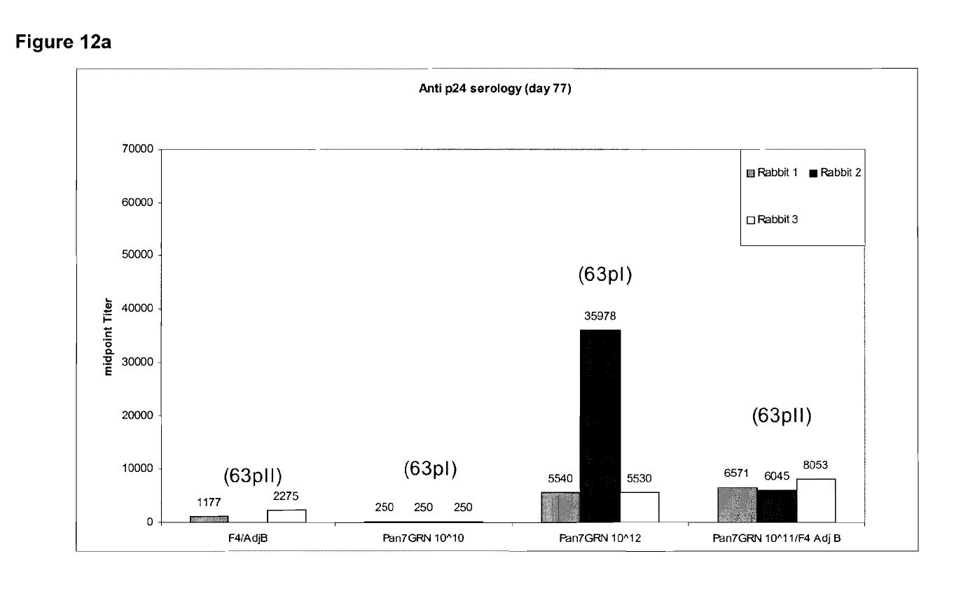

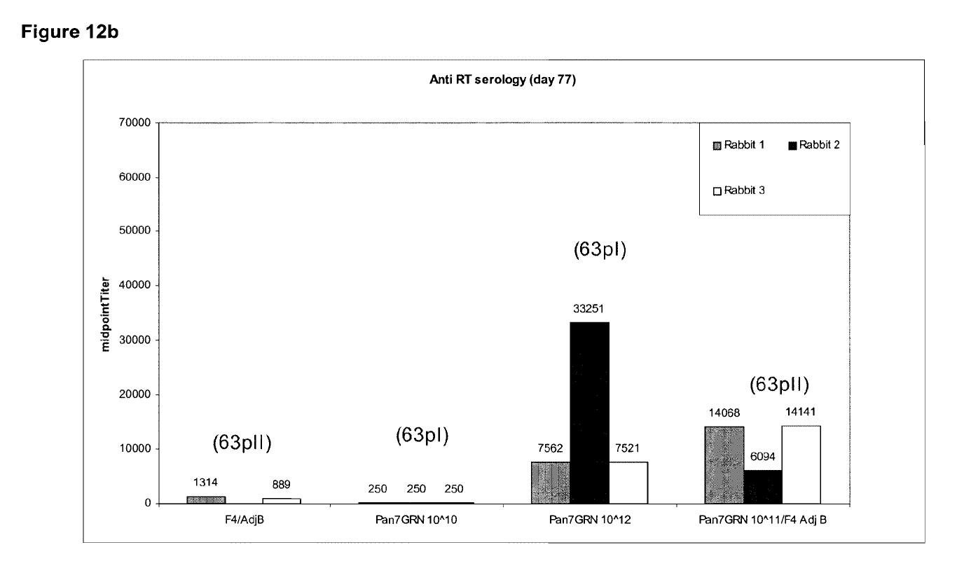

As shown in FIG. 11, the kinetic of the anti-F4 antibody response observed in the animals immunised twice with the combination of adenovirus/protein/adjuvant is similar to the one from animals immunised twice with the protein: it is already detected at 7 days post-2^{nd} injection and then decrease over time. However, in terms of intensity, the anti-F4 response of animals immunised twice with the combination of adenovirus/protein/adjuvant remains higher at later timepoints (21 and 63 days post-2^{nd} immunisation) when compared to the anti-F4 response from animals immunised twice with the protein. No anti-F4 antibody response is observed in rabbits immunised once with 10¹⁰ viral particles of adenovirus. In rabbits immunised once with 10¹² viral particles of adenovirus, an anti-F4 response is only detected at 21 and 63 days post-immunisation. In that group, the high variability of the response observed at the 63 day post-immunisation timepoint (d77) results from a single animal (out of the 3) displaying higher titers against the different F4 components, especially p24 and RT as shown in FIGS. 12a and 12b respectively. The anti-F4 antibody response is mainly composed of antibodies targeting p24 and RT and to a much lesser extent Nef and p17.

### Conclusion:

Lymphoproliferative and antibody responses could be induced in rabbits after two injections of a composition containing an immunogenic polypeptide, an adenoviral vector containing a heterologous polynucleotide encoding an immunogenic polypeptide and an adjuvant. In addition, we have evidence that a lymphoproliferative response can be recalled after a third injection of such composition. Finally, the best antibody response (in intensity and persistence) is observed with the adenovirus/protein/adjuvant combination.

### Example 4

### Immunogenicity of F4 (Codon Optimized)/Adjuvant B and C7-GRN when Administrated as a Combination in CB6F1 Mice

### Experimental Design

CB6F1 mice were immunized twice (days 0 and 21) with different combinations listed below. F4co/adjuvant B was used at 9 μg F4co/animal in 50 μl AdjuvantB (1/10 human dose) and the C7-GRN virus at 108 viral particles/animal. F4co in Example 4 is F4 prepared as described in WO2006/013106 Example 1, codon-optimised method.

### Combinations

C7-GRN
C7-GRN/adjuvant B
C7-GRN/F4co
C7-GRN/F4co/adjuvant B
F4co
F4co/adjuvant B
adjuvant B
C7 empty
C7empty/adjuvant B
C7empty/F4co
C7empty/F4col adjuvant B

### Schedule of Immunizations and Immune Response Analysis

Immunisations were carried out at day 0 and day 21. Intracellular cytokine staining (ICS) was carried out at 21 days, 28 days (7 days post immunisation 2), 42 days (21 days post immunisation 2), and 77 days (56 days post immunisation 2).

### Results

### HIV-Specific CD4 T Cell Responses

The results are shown in the following figures:

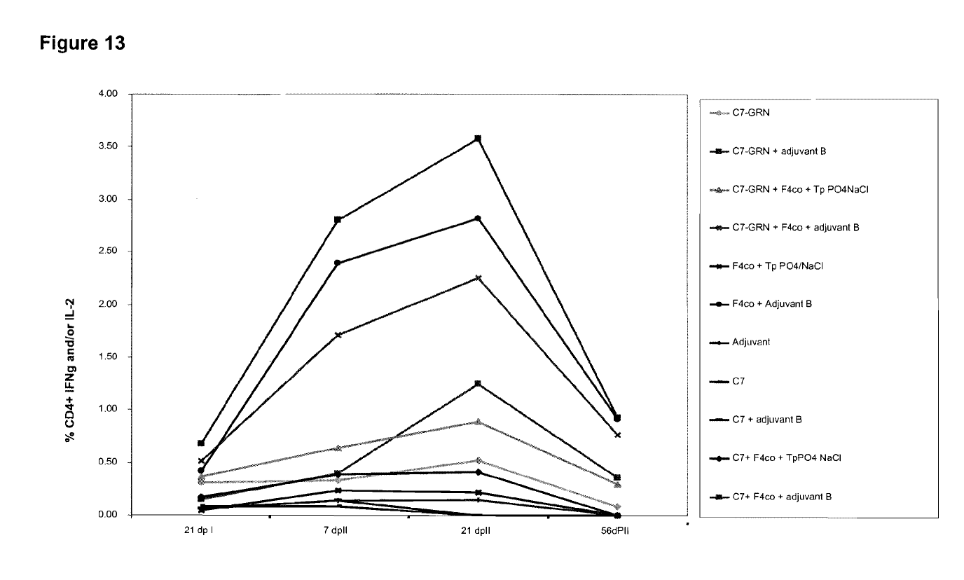

FIG. 13. Quantification of HIV-1-specific CD4 T cells. The % of CD3 CD4 T cells secreting IFN-γ and/or IL-2 is represented for each protocol of immunization at four time-points. Peripheral blood lymphocytes (PBLs) were stimulated ex vivo (2 hours before addition of the Brefeldin then overnight) with a pool of peptides covering F4 sequence and the cytokine production was measured by ICS. Each value is the geometric mean of 5 pools of 3 mice.

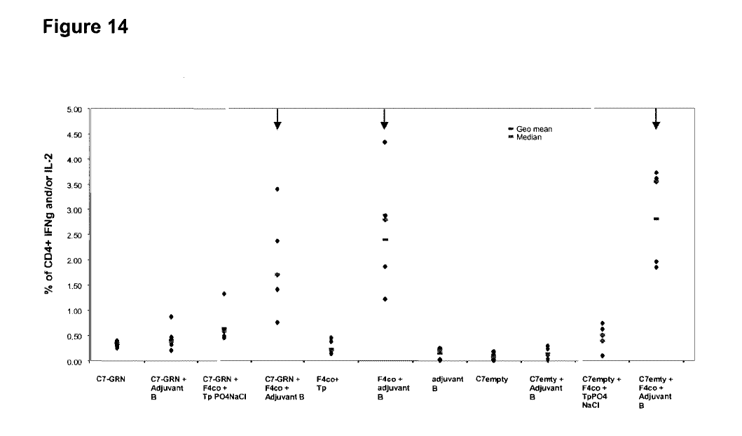

FIG. 14. Distribution of the frequency of F4-specific CD4 T cells 7 days after two immunizations. The frequency of F4-specific circulating CD4 T cells at 7 days after two immunizations is represented for each protocol. Each dot represents the value obtained for one pool of 3 mice.

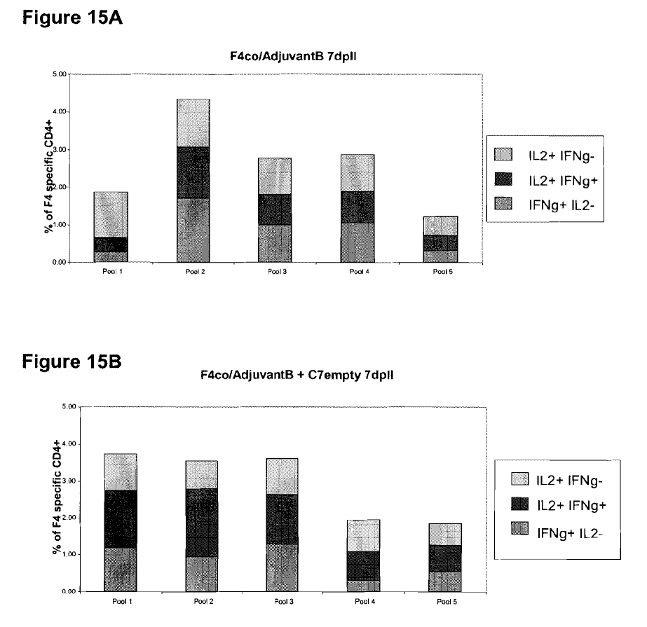

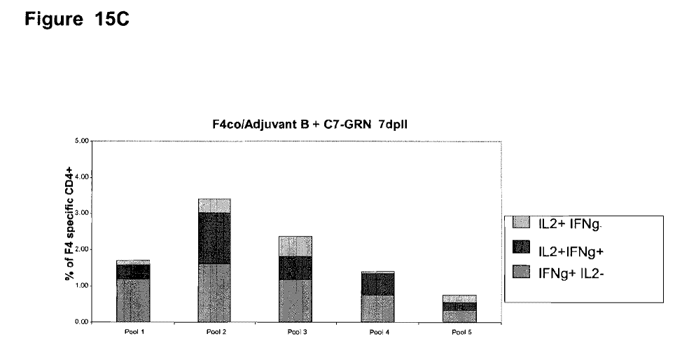

FIG. 15. Cytokine production of F4-specific CD4 T cells 7 days after two immunizations. The % of F4-specific CD4 T cells secreting IL-2 and/or IFN-γ is represented for 5 pools of 3 mice. Results for the immunization with F4co/adjuvant B (A), F4co/adjuvant B/C7 empty (B) and F4co/adjuvant B/C7-GRN(C) are presented.

The frequency of F4-specific circulating CD4 T cells reaches 2.82% 21 days after two immunizations with the F4co/adjuvant B combination and declines to 0.91% 56 days post-immunization (FIG. 13). Two doses of the C7-GRN virus alone result in 0.52% of F4-specific circulating CD4 T cells 21 days post last immunization and the presence of the adjuvant adjuvant B does not alter this response.

The presence of the empty vector C7 or the recombinant C7-GRN virus in addition of the F4co/adjuvant B mix does not increase nor interfere with the frequency of F4-specific CD4 T cell response (3.58% and 2.82% respectively, 21 days post-last immunization). Even if no statistical analysis has been performed, the population distribution suggests that the intensity of the F4-specific CD4 T cell responses is not different between the three protocols F4col adjuvant B, F4co/adjuvant B/C7 empty and F4co/adjuvant B/C7-GRN (FIG. 14).

As expected, administration of the F4co without adjuvant B does not induce significant F4-specific CD4 T cells.

The profile of cytokine production shows that after immunization with F4co/adjuvant B, the F4-specific CD4 T cells secrete both IFN-γ and IL-2. Addition of C7empty or C7-GRN in the immunization protocol does not alter this profile.

As a result, these data suggest that the greatest F4-specific CD4 T cell response is obtained after immunization with the F4co/adjuvant B combination and that the presence of the C7-GRN virus does not improve nor alter this response.

### Antigen-Specific CD8 T Cell Responses

The results are shown in the following figures

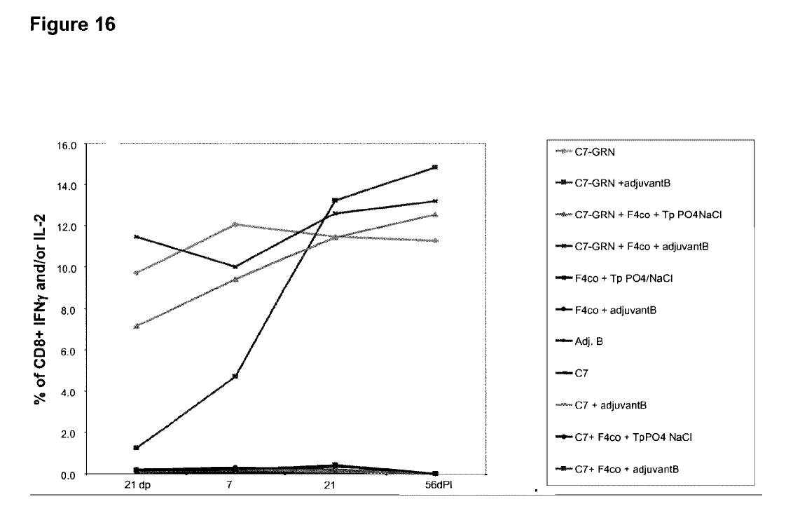

FIG. 16. Quantification of HIV-1-specific CD8 T cells. The % of CD3 CD8 T cells secreting IFN-γ and/or IL-2 is represented for each protocol of immunization at four time-points. Peripheral blood lymphocytes (PBLs) were stimulated ex vivo (2 hours before addition of Brefeldin then overnight) with a pool of peptides covering F4 and the cytokine production was measured by ICS. Each value is the geometric mean of 5 pools of 3 mice.

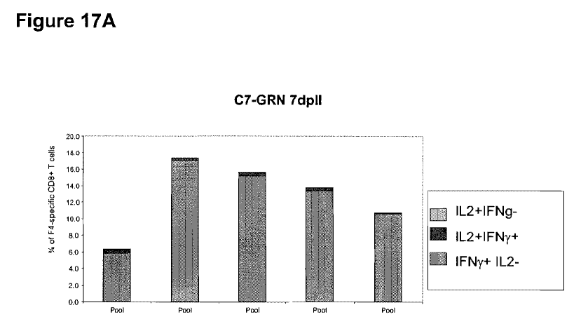

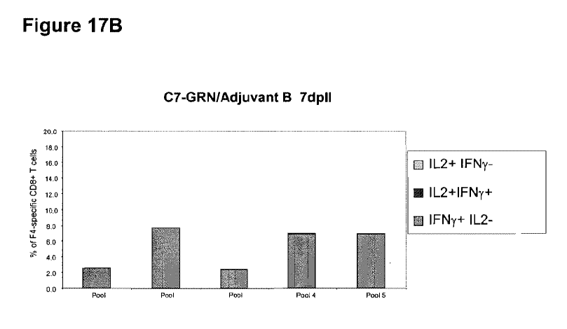

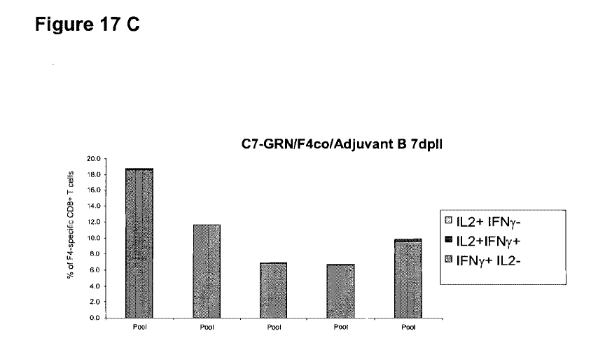

FIG. 17. Cytokine production of F4-specific CD8 T cells 7 days after two immunizations. The % of F4-specific CD8 T cells secreting IL-2 and/or IFN-γ is represented for 5 pools of 3 mice. Results for the immunization with C7-GRN (A), C7-GRN/adjuvant B (B) and C7-GRN+F4co/adjuvant B (C) are presented.

After one injection, the recombinant vector C7-GRN induces a high frequency of F4-specific circulating CD8 T cells (9.70% of total CD8 T cells, 21 days post-immunization) (FIG. 4). A second injection does not boost the F4-specific CD8 T cell response. The F4co/adjuvant B combination induces low to undetectable F4-specific CD8 T cells and adding this combination to the C7-GRN does not improve or impair the F4-specific CD8 T cell response.

The F4-specific CD8 T cell response is delayed when the adjuvant B is added to the C7-GRN, but reaches the same level as with the C7-GRN alone or the C7-GRN/F4co/adjuvant B combination at 21 days post-second immunization.

The F4-specific CD8 T cells mainly secrete IFN-γ whether the C7-GRN vector is injected alone or in combination with F4co/adjuvant B (FIG. 17).

Interestingly, the F4-specific CD8 T cell response persists up to 56 days post-last immunization without declining, suggesting that the C7 vector elicits high and persistent CD8 T cells.

### Conclusions

The F4co/adjuvant B vaccine induces a high frequency of poly-functional HIV-specific CD4 T cells but no HIV-specific CD8 T cells in CB6F1 mice. In the same animal model, the recombinant adenovirus C7 expressing Gag, RT and Nef (Ad C7-GRN) induces a high antigen-specific CD8 T cell response and low to undetectable antigen-specific CD4 T cells. A combination of F4/adjuvant B and Ad C7-GRN elicits both antigen-specific CD4 and CD8 T cells at the same time. A combination of three components, F4co, adjuvantB and C7-GRN elicits the highest levels of both antigen specific CD4 and CD8 T cells at the same time. Combining F4/adjuvant B and Ad C7-GRN has an additive effect concerning the intensity of both arms of the cellular immune response. The effect of the antigen-specific CD4 T cell response on the functionality of antigen-specific CD8 T cell response remains to be determined in this model.

### Example 5

### Immunogenicity of the Chimpadenovirus C7 expressing CS2 construct of CSP Protein from Plasmodium falciparum (C7-CS2) when Administered Alone

### Experimental Design:

CB6F1 mice were immunized once intramuscularly with a dose range (10¹⁰, 10⁹ & 10⁸ viral particles) of the C7 chimpadenovirus expressing the CSP malaria antigen and the CSP-specific (C-term and N-term) CD4 and CD8 T cell responses were determined 21, 28 and 35 days post-injection by ICS (Intra-cellular Cytokine Staining).

### CSP-Specific CD4 T Cell Responses

The results are shown in the following figures:

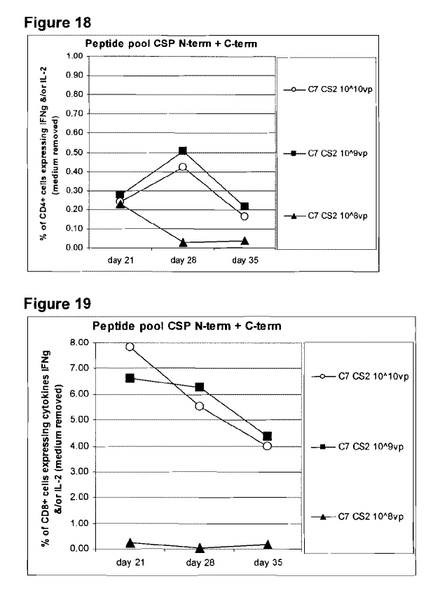

FIG. 18. Quantification of CSP-specific CD4 T cells. The % of CD4 T cells secreting IFN-γ and/or IL-2 is represented for each protocol of immunization at three time-points. Peripheral blood lymphocytes (PBLs) were stimulated ex vivo (2 hours before addition of the Brefeldin then overnight) with a pool of peptides covering CSP N-term or CSP C-term sequences and the cytokine production was measured by ICS. The responses to the C-term and N-term peptide pools were added up and each value is the average of 5 pools of 4 mice.

FIG. 19. Quantification of CSP-specific CD8 T cells. The % of CD8 T cells secreting IFN-γ and/or IL-2 is represented for each protocol of immunization at three time-points. Peripheral blood lymphocytes (PBLs) were stimulated ex vivo (2 hours before addition of the Brefeldin then overnight) with a pool of peptides covering CSP N-term or CSP C-term sequences and the cytokine production was measured by ICS. The responses to the C-term and N-term peptide pools were added up and each value is the average of 5 pools of 4 mice.

These results indicate that both 10¹⁰ and 10⁹ doses of C7-CS2 elicit similar levels of CSP-specific CD4 T cell responses (peak 0.5%) and similar levels of CSP-specific CD8 T cell responses (peak 8%). The dose of 10¹⁰ of C7-CS2 was chosen in subsequent experiments where the immunogenicity of C7-CS2 in combination with RTS,S was tested (see below).

### Example 6

### Immunogenicity of C7-CS2 and RTS,S when Administered as a Combination in CB6F1 Mice

### Experimental Design:

CB6F1 mice were immunized three times intramuscularly (day 0, 14 & 28) with either a combination of the malaria vaccine candidate RTS,S (5 μg) in 50 μl of Adjuvant B (referred as P—P—P in the figures below) or a combination of RTS,S (5 μg) and C7-CS2(10¹⁰ viral particles) in 50 μl of Adjuvant B (referred as C—C—C in the figures below). The CSP-specific (C-term and N-term) CD4 and CD8 T cell responses were determined at the following time-points:
7 days post 2 immunizations
7, 21, 35 and 49 days post 3 immunizations

CSP-specific T cell responses were determined by ICS (Intra-cellular Cytokine Staining).

The CSP-specific antibody responses in the sera from immunized animals were also determined by ELISA at 14 and 42 days post-3^{rd} immunization.

### CSP-Specific CD4 T Cell Responses

The results are shown in the following figures:

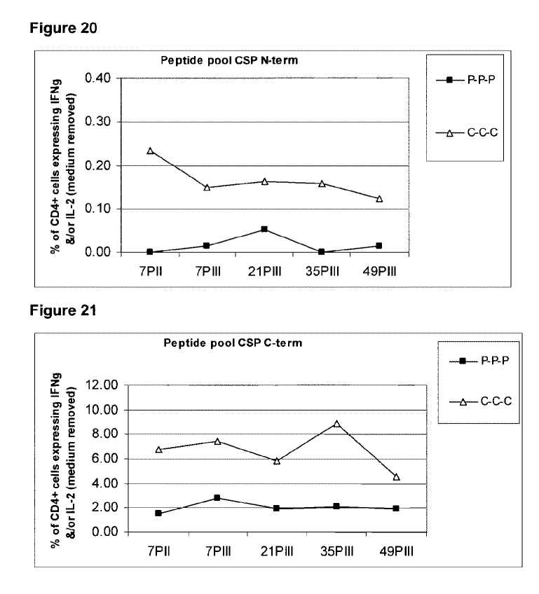

FIG. 20. Quantification of CSP(N-term)-specific CD4 T cells. The % of CD4 T cells secreting IFN-γ and/or IL-2 is represented for each protocol of immunization at five time-points. Peripheral blood lymphocytes (PBLs) were stimulated ex vivo (2 hours before addition of the Brefeldin then overnight) with a pool of peptides covering the CSP N-term sequence and the cytokine production (IFNg and/or IL-2) was measured by ICS. Each value is the average of 4 pools of 7 mice.

FIG. 21. Quantification of CSP(C-term)-specific CD4 T cells. The % of CD4 T cells secreting IFN-γ and/or IL-2 is represented for each protocol of immunization at five time-points. Peripheral blood lymphocytes (PBLs) were stimulated ex vivo (2 hours before addition of the Brefeldin then overnight) with a pool of peptides covering the CSP C-term sequence and the cytokine production (IFNg and/or IL-2) was measured by ICS. Each value is the average of 4 pools of 7 mice.

These results indicate that mice immunized with 3 injections of the combination [RTS,S+C7-CS2 10¹⁰+Adjuvant B] display higher antigen-specific CD4 T cell responses (both against the C-term and N-term part of CSP) than the mice immunized with 3 injections of RTS,S+Adjuvant B.

### CSP-Specific CD8 T Cell Responses

The results are shown in the following figures:

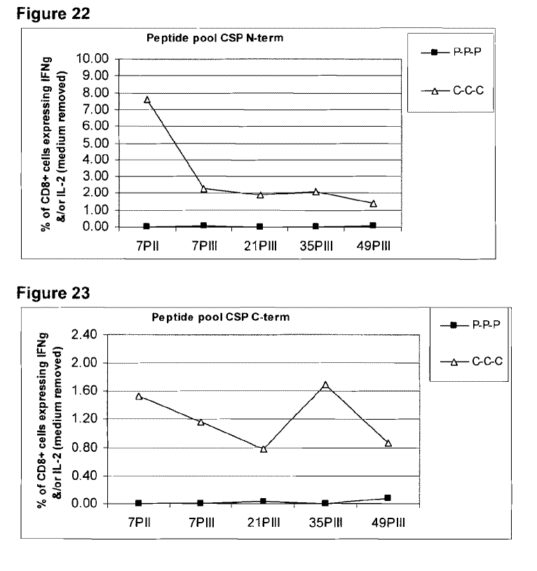

FIG. 22. Quantification of CSP(N-term)-specific CD8 T cells. The % of CD8 T cells secreting IFN-γ and/or IL-2 is represented for each protocol of immunization at five time-points. Peripheral blood lymphocytes (PBLs) were stimulated ex vivo (2 hours before addition of the Brefeldin then overnight) with a pool of peptides covering the CSP N-term sequence and the cytokine production (IFNg and/or IL-2) was measured by ICS. Each value is the average of 4 pools of 7 mice.

FIG. 23. Quantification of CSP(C-term)-specific CD8 T cells. The % of CD8 T cells secreting IFN-γ and/or IL-2 is represented for each protocol of immunization at five time-points. Peripheral blood lymphocytes (PBLs) were stimulated ex vivo (2 hours before addition of the Brefeldin then overnight) with a pool of peptides covering the CSP C-term sequence and the cytokine production (IFNg and/or IL-2) was measured by ICS. Each value is the average of 4 pools of 7 mice.

These results indicate that mice immunized with 3 injections of the combination [RTS,S+C7-CS2 10¹⁰±Adjuvant B] display higher antigen-specific CD8 T cell responses (both against the C-term and N-term part of CSP) than the mice immunized with 3 injections of RTS,S+Adjuvant B.

### CSP-Specific Antibody Responses

The results are shown in the following figure:

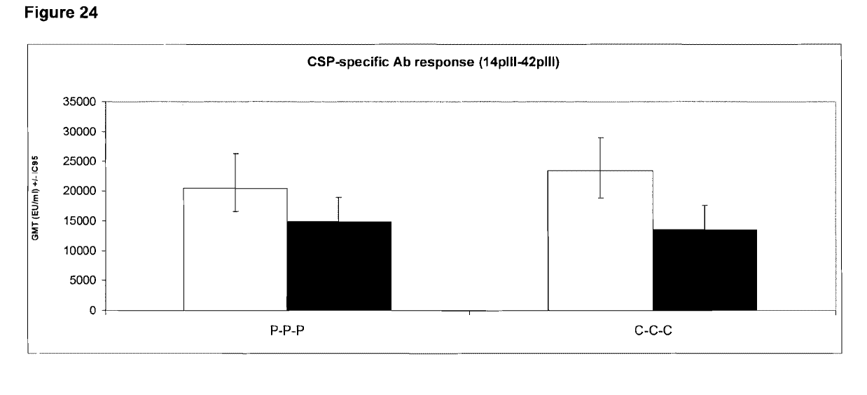

FIG. 24. Quantification of CSP-specific antibody titers. The sera from the mice were collected at 14 and 42 days post 3^{rd} immunization. The anti-CSP antibody titers were measured in each of these individual sera by ELISA. The data shown is the geometric mean antibody titers±95% confidence interval.

These results indicate that mice immunized with 3 injections of the combination [RTS,S+C7-CS2 10¹⁰+Adjuvant B] display similar CSP-specific antibody titers than the mice immunized with 3 injections of RTS,S+Adjuvant B.

### Conclusions

The RTS,S/adjuvant B vaccine induces a high frequency of CSP C-term-specific CD4 T cells but no CSP N-term specific CD4 T cells. In addition, the RTS,S/adjuvant B vaccine induces low to undetectable CSP C& N-term specific CD8 T cells. In the same animal model, the recombinant adenovirus C7 expressing CSP induces high CSP(C-term and N-term)-specific CD8 T cell responses and lower CSP(C-term and N-term)-specific CD4 T cell responses. A combination of RTS,S/adjuvant B and Ad C7-CS2 elicits high levels of both CSP(C-term and N-term)-specific CD4 and CD8 T cells at the same time. Combining RTS,S/adjuvant B and Ad C7-CS2 has an additive effect concerning the intensity of both arms of the T cell response. Finally, the combination of RTS,S/adjuvant B and Ad C7-CS2 elicits high levels of CSP-specific antibody responses that are comparable to the ones induced by RTS,S/adjuvant B.

All references referred to in this application, including patent and patent applications, are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1.-7. (canceled)

8. A composition comprising (i) one or more first immunogenic polypeptides from a pathogen; (ii) one or more adenoviral vectors comprising one or more heterologous polynucleotide encoding one or more second immunogenic polypeptides from the pathogen; and (iii) an adjuvant.

9. The composition of claim 8, wherein one or more of the one or more first immunogenic polypeptides is substantially the same as one or more of the one or more second immunogenic polypeptides.

10. The composition of claim 8, wherein one or more of the one or more first immunogenic polypeptides comprises at least one antigen which is substantially the same as an antigen in one or more of the one or more second immunogenic polypeptides.

11. The composition of claim 8, wherein one or more of the first immunogenic polypeptides comprises at least one T cell epitope.

12. The composition of claim 8, wherein one or more of the first immunogenic polypeptides comprises at least one B cell epitope.

13.-14. (canceled)

15. The composition of claim 8, wherein one or more of the adenoviral vectors is produced from a human adenovirus.

16. The composition of claim 15, wherein the human adenovirus serotype is selected from Ad1, Ad2, Ad4, Ad5, Ad6, Ad11, Ad 24, Ad34 and Ad35.

17. The composition of claim 8, wherein one or more of the adenoviral vectors is produced from a non-human primate adenovirus.

18. The composition of claim 17, wherein the non-human primate adenovirus serotype is selected from chimpanzee adenovirus serotypes Pan5, Pan6, Pan7 and Pan9.

19. The composition of claim 8, wherein the pathogen is HIV.

20. The composition of claim 19, wherein the immunogenic polypeptides contain HIV derived antigens which are selected from Env, Nef, Gag, and Pol and immunogenic derivatives thereof and immunogenic fragments thereof.

21. The composition of claim 20, wherein a first immunogenic polypeptide is p24-RT-Nef-p17.

22. The composition of claim 20, wherein a second immunogenic polypeptide is Gag-RT-Nef.

23. The composition of claim 8, wherein the pathogen is Plasmodium falciparum and/or Plasmodium vivax.

24. The composition of claim 23, wherein the immunogenic polypeptides contain antigens from Plasmodium falciparum and/or Plasmodium vivax which are selected from circumsporozoite (CS) protein, MSP-1, MSP-3, AMA-1, LSA-1, LSA-3, and immunogenic fragments thereof.

25. The composition of claim 24, wherein a/the immunogenic polypeptide comprises the hybrid protein RTS.

26.-27. (canceled)

28. The composition of claim 8, wherein the pathogen is Mycobacterium tuberculosis.

29. The composition of claim 8, wherein the adjuvant comprises a preferential stimulator of Th1 responses.

30. The composition of claim 29, wherein the adjuvant comprises at least one of QS21, 3D-MPL and CpG.

31. The composition of claim 30 wherein the adjuvant comprises QS21 and 3D-MPL.

32. The composition of claim 8, wherein the adjuvant contains an oil-in-water emulsion.

33. The composition of claim 8, wherein the adjuvant contains liposomes.

34. A method of stimulating an immune response in a mammal comprising administering to a subject an immunologically effective amount of the composition of claim 8.

35.-36. (canceled)

37. A kit comprising (i) one or more first immunogenic polypeptides derived from a pathogen; (ii) one or more adenoviral vectors comprising one or more heterologous polynucleotides encoding one or more second immunogenic polypeptides derived from said pathogen; and (iii) an adjuvant.

38. The kit of claim 37, wherein the kit comprises a composition comprising the one or more first immunogenic polypeptides and an adjuvant.

39. The composition of claim 8, wherein the first immunogenic polypeptide comprises p24-RT-Nef-p17, the adjuvant comprises 3D-MPL and QS21 in a liposome, and the adenoviral vector comprises a chimpanzee adenovirus serotype Pan7 vector comprising a polynucleotide encoding the immunogenic polypeptide Gag-RT-Nef.

40. (canceled)

41. A method of raising an immune response against a pathogen comprising administering (i) one or more first immunogenic polypeptides from said pathogen; (ii) one or more adenoviral vectors comprising one or more heterologous polynucleotides encoding one or more second immunogenic polypeptides from said pathogen; and (iii) an adjuvant, wherein the one or more first immunogenic polypeptides, the one or more adenoviral vectors and the adjuvant are administered concomitantly.

42. The method of claim 41, wherein the one or more first immunogenic polypeptides derived from said pathogen are co-formulated with the adjuvant.

43. The method of claim 41, wherein the administering stimulates the production of one or more of pathogen-specific CD4+ T cells, CD8+ T-cells and antibodies.

44. The method of claim 41, wherein the administering is repeated.

45. The method of claim 41, wherein the method does not involve administering any priming dose of immunogenic polypeptide or polynucleotide encoding immunogenic polypeptide.

46. The method of claim 41, wherein the one or more immunogenic polypeptides, the one or more adenoviral vectors and the adjuvant are co-formulated.
